(19) United States
(12) Patent Application Publication
(10) Pub. No.: US 2009/0082570 A1
(43) Pub. Date: Mar. 26, 2009

(54) METHOD OF MANUFACTURING BIPYRIDINIUM COMPOUND AND SYNTHETIC INTERMEDIATE OF THE SAME, METHOD OF MANUFACTURING DYE COMPOUND, AND NOVEL BIPYRIDINIUM COMPOUND AND NOVEL DYE COMPOUND COMPRISING THE SAME

(75) Inventors: Kazumi Nii, Kanagawa (JP); Toshinao Ukai, Kanagawa (JP); Kazufumi Omura, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/212,023

(22) Filed: Sep. 17, 2008

(30) Foreign Application Priority Data
Sep. 20, 2007 (JP) 2007-243189

(51) Int. Cl. C07D 401/04

(52) U.S. Cl. 546/258; 546/257

(57) ABSTRACT

Provided is a method of manufacturing a bipyridinium compound denoted by general formula (A).

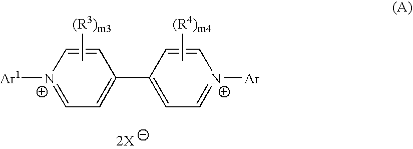

In general formula (A), Ar¹ and Ar² each independently denote an optionally substituted (hetero)aryl group, R³ and R⁴ each independently denote a substituent that may form a ring with a pyridine ring to which the substituent substitutes, m3 and m4 each independently denote an integer ranging from 0 to 4, X denotes a halogen atom or RSO₃, and R denotes an optionally substituted aryl group or alkyl group. The method can manufacture a 4,4′-bipyridinium compound under mild reaction conditions in an integrated manner without separation of intermediates.

### CROSS-REFERENCE TO RELATED APPLICATIONS

This application claims the benefit of priority under 35 USC 119 to Japanese Patent Application No. 2007-243189 filed on Sep. 20, 2007, which is expressly incorporated herein by reference in its entirety.

### BACKGROUND OF THE INVENTION

1. Field of the Invention

The present invention relates to a method of manufacturing 4,4′-bipyridinium compounds, which are useful as herbicides, electrochromic display materials, functional dyes such as optical recording-use dyes, and their constituent materials; a method of manufacturing synthetic intermediates that can be used in the above manufacturing method; and a method of manufacturing dye compounds comprising the 4,4′-bipyridinium compounds obtained by the above manufacturing method.

The present invention further relates to a novel bipyridinium compound and a novel dye compound comprising the same.

2. Discussion of the Background

4,4′-bipyridinium compounds have been used in herbicides. In recent years, they have also been examined for use as electrochromic display materials.

One known method of manufacturing 4,4′-bipyridinium compounds is called the Menshutkin reaction. However, 4,4′-bipyridinium compounds in the form of aryl-substituted derivatives cannot be produced by the Menshutkin reaction.

The method indicated below is an example of a method of manufacturing aryl-substituted 4,4′-bipyridinium compounds. Such method is disclosed in Bull. Chem. Soc. Jpn., 1991, Vol. 64, pp. 321-323, which is expressly incorporated herein by reference in its entirety.

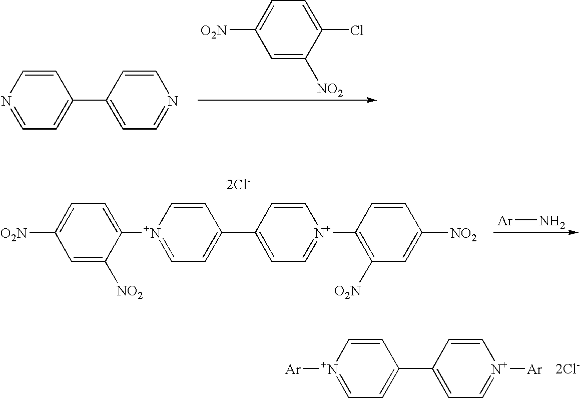

Another method of manufacturing aryl-substituted 4,4′-bipyridinium compounds has been proposed in the form of a method the key of which is reacting 4,4′-bipyridine with a (hetero)arylhalogen compound. Such method is proposed in Japanese Unexamined Patent Publication (KOKAI) No. 2003-128654, which is expressly incorporated herein by reference in its entirety.

The burden placed on the environment by chemical product manufacturing processes has become an issue in recent years. There has been a call for clean chemical reactions with mild reaction conditions, placing little burden on the environment or operations, in which a minimum of harmful solvents, reactants, and the like are employed. Under such conditions, Japanese Unexamined Patent Publication (KOKAI) No. 2005-314377 or English language family member US 2006/0258870 A1, which are expressly incorporated herein by reference in their entirety, discloses an example of a method of manufacturing an aryl-substituted bipyridinium compound employing a polyhydric alcohol.

However, the method described in Japanese Unexamined Patent Publication (KOKAI) No. 2005-314377 can only be used to manufacture symmetric bipyridinium compounds in which the two aryl groups that are substituents on the nitrogen of the bipyridinium are identical. It cannot be used to synthesize asymmetric bipyridinium compounds with two different aryl groups.

### SUMMARY OF THE INVENTION

An aspect of the present invention provides for a synthesis method permitting the synthesis of aryl-substituted bipyridinium compounds, desirably asymmetrically aryl-substituted bipyridinium compounds, by a clean method in which a minimum of harmful solvents, reactants, and the like are employed, that places little burden on the environment or on operations, and can be conducted quickly and under mild reaction conditions.

We conducted extensive research into obtaining the above method, resulting in the discovery that, surprisingly, the use of specific reaction conditions tempered the reaction and permitted the integrated manufacturing of 4,4′-bipyridinium compounds without the separation of intermediates, and that this method permitted the manufacturing of asymmetric aryl-substituted bipyridinium compounds. The present invention was devised on that basis.

An aspect of the present invention relates to a method of manufacturing a bipyridinium compound denoted by general formula (A) comprising steps of:

(a) manufacturing a bipyridinium compound denoted by general formula (3) by reacting a bipyridine compound denoted by general formula (1) with a (hetero)arylhalogen compound denoted by general formula (2) in a solvent;

(b) manufacturing a N-aryl-substituted bipyridinium compound denoted by general formula (5) by reacting the bipyridinium compound denoted by general formula (3) that has been obtained in step (a) with an amine compound denoted by general formula (4) in a solvent;

(c) manufacturing a bipyridinium compound denoted by general formula (7) by reacting the N-aryl-substituted bipyridinium compound denoted by general formula (5) that has been obtained in step (b) with a (hetero)arylhalogen compound denoted by general formula (6) in a solvent; and

(d) manufacturing the bipyridinium compound denoted by general formula (A) by reacting the bipyridinium compound denoted by general formula (7) that has been obtained in step (c) with an amine compound denoted by general formula (8) in a solvent.

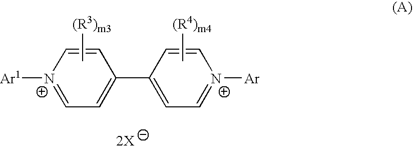

In general formula (A), Ar¹ and Ar² each independently denote an optionally substituted (hetero)aryl group, R³ and R⁴ each independently denote a substituent that may form a ring with a pyridine ring to which the substituent substitutes, m3 and m4 each independently denote an integer ranging from 0 to 4, X denotes a halogen atom or RSO₃, and R denotes an optionally substituted aryl group or alkyl group. When m3 denotes an integer ranging from 2 to 4, plural R³ present may be identical or different from each other and when m4 denotes an integer ranging from 2 to 4, plural R⁴ present may be identical or different from each other.

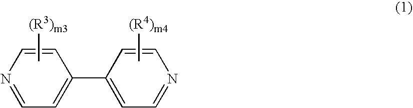

In general formula (1), R³, R⁴, m3 and m4 are respectively defined as in general formula (A).

R¹—X (2)

In general formula (2), R¹ denotes an optionally substituted (hetero)aryl group, and X is defined as in general formula (A).

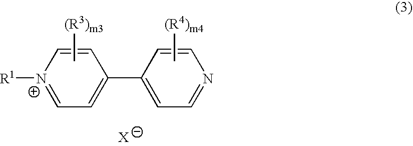

In general formula (3), R¹, R³, R⁴, m3, m4 and X are respectively defined as in general formula (A) or (2).

Ar¹—NH₂ (4)

In general formula (4), Ar¹ is defined as in general formula (A).

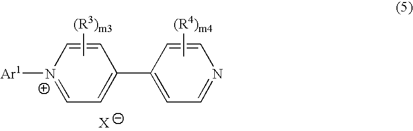

In general formula (5), Ar¹, R³, R⁴, m3, m4 and X are respectively defined as in general formula (A).

R²—X (6)

In general formula (6), R² denotes an optionally substituted (hetero)aryl group, and X is defined as in general formula (A).

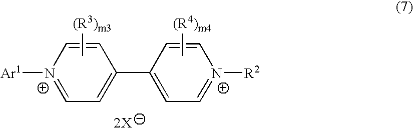

In general formula (7), Ar¹, R², R³, R⁴, m3, m4 and X are respectively defined as in general formula (A) or (6).

Ar²—NH₂ (8)

In general formula (8), Ar² is defined as in general formula (A).

Ar¹ and Ar² may be different from each other.

The above method may comprise no purification step between step (a) and step (b) and between step (c) and step (d).

The above method may comprise an extraction step with water between step (b) and step (c).

In the above method, the solvent may comprise at least one selected from the group comprising of acetonitrile, an amide solvent, and an alcohol solvent.

A further aspect of the present invention relates to a method of manufacturing a bipyridinium compound denoted by general formula (7) comprising steps of the above (a) to (c).

The above method may comprise an extraction step with water between step (b) and step (c).

In the above method, the solvent may comprise at least one selected from the group comprising of acetonitrile, an amide solvent, and an alcohol solvent.

A further aspect of the present invention relates to a method of manufacturing a bipyridinium compound denoted by general formula (B) comprising steps of:

(a) manufacturing a bipyridinium compound denoted by general formula (3) by reacting a bipyridine compound denoted by general formula (1) with a (hetero)arylhalogen compound denoted by general formula (2) in a solvent;

(b) manufacturing a N-aryl-substituted bipyridinium compound denoted by general formula (5) by reacting the bipyridinium compound denoted by general formula (3) that has been obtained in step (a) with an amine compound denoted by general formula (4) in a solvent;

(c) manufacturing a bipyridinium compound denoted by general formula (7) by reacting the N-aryl-substituted bipyridinium compound denoted by general formula (5) that has been obtained in step (b) with a (hetero)arylhalogen compound denoted by general formula (6) in a solvent; and

(e) manufacturing the bipyridinium compound denoted by general formula (B) by reacting the bipyridinium compound denoted by general formula (7) that has been obtained in step (c) with a diamine compound denoted by general formula (9) in a solvent.

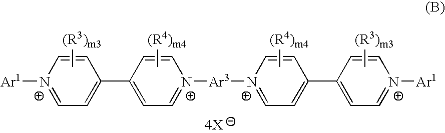

In general formula (B), Ar³ denotes an optionally substituted (hetero)arylene group, and Ar¹, R³, R⁴, m3, m4 and X are respectively defined as in general formula (A).

H₂N—Ar³—NH₂ (9)

In general formula (9), Ar³ is defined as in general formula (B).

The above method may comprise no purification step between step (a) and step (b) and between step (c) and step (e).

The above method may comprise an extraction step with water between step (b) and step (c).

In the above method, the solvent may comprise at least one selected from the group comprising of acetonitrile, an amide solvent, and an alcohol solvent.

A further aspect of the present invention relates to a method of manufacturing a dye compound denoted by general formula (D) comprising:

manufacturing a bipyridinium compound denoted by general formula (A) by the above manufacturing method; and

manufacturing the dye compound denoted by general formula (D) by reacting the bipyridinium compound that has been obtained with an anionic dye.

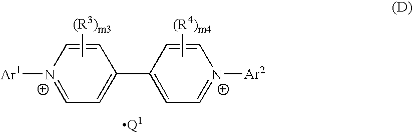

In general formula (D), Q¹ denotes a divalent anionic dye moiety, and Ar¹, Ar², R³, R⁴, m3 and m4 are respectively defined as in general formula (A).

The divalent anionic dye moiety may be an oxonol dye denoted by general formula (10).

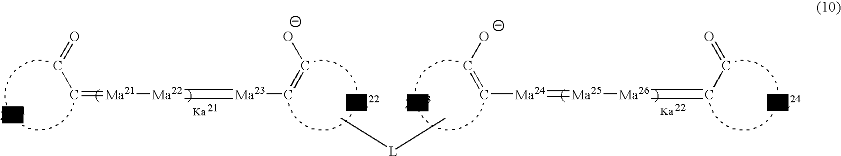

In general formula (10), Za²¹, Za²², Za²³ and Za²⁴ each independently denote an atom group forming an acidic nucleus, Ma²¹, Ma²², Ma²³, Ma²⁴, Ma²⁵ and Ma²⁶ each independently denote a substituted or unsubstituted methine group, L denotes a divalent linking group that does not form a π-conjugated system with two bonds, Ka²¹ and Ka²² each independently denote an integer ranging from 0 to 3. When Ka²¹ denotes 2 or 3, plural Ma²¹ and Ma²² present may be identical or different from each other, and when Ka²² denotes 2 or 3, Ma²⁵ and Ma²⁶ present may be identical or different from each other.

A further aspect ofthe present invention relates to a method of manufacturing a dye compound denoted by general formula (E) comprising:

manufacturing a bipyridinium compound denoted by general formula (B) by the above manufacturing method; and

manufacturing the dye compound denoted by general formula (E) by reacting the bipyridinium compound that has been obtained with an anionic dye.

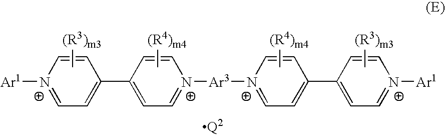

In general formula (E), Q² denotes two divalent anionic dye moieties, and Ar¹, Ar³, R³, R⁴, m3 and m4 are respectively defmed as in general formula (B).

The divalent anionic dye moiety may be an oxonol dye denoted by general formula (10).

A further aspect of the present invention relates to a compound denoted by general formula (C) or a salt thereof.

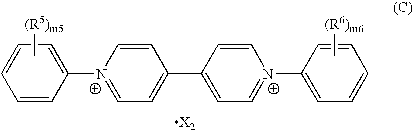

In general formula (C), R⁵ and R⁶ each independently denote a substituent that may form a ring with a benzene ring to which the substituent substitutes, m5 and m6 each independently denote an integer ranging from 0 to 5, and X² denotes an anion that neutralizes a charge within a molecule. When m5 denotes an integer ranging from 2 to 5, plural R⁵ present may be identical or different from each other and when m6 denotes an integer ranging from 2 to 5, plural R⁶ present may be identical or different from each other. m5 and m6 do not both denote 0, and the substituent group denoted by (R⁵)m5 and the substituent group denoted by (R⁶)m6 are not identical.

A further aspect of the present invention relates to a dye compound denoted by general formula (F).

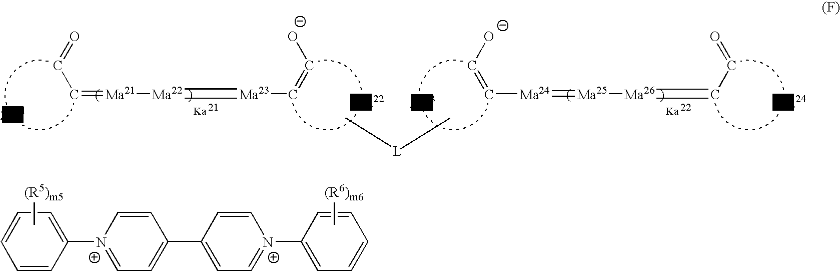

In general formula (F), Za²¹, Za²², Za²³, Za²⁴, Ma²¹, Ma²², Ma²³, Ma²⁴, Ma²⁵, Ma²⁶, L, Ka²¹ and Ka²² are respectively defined as in general formula (10), and R⁵, R⁶, m5 and m6 are respectively defined as in general formula (C).

The present invention permits the safe, efficient, and economical manufacturing on an industrial scale of 4,4′-bipyridinium compounds that are useful as herbicides, electrochromic display materials, optical recording materials, and their constituent materials.

Other exemplary embodiments and advantages of the present invention may be ascertained by reviewing the present disclosure.

### DETAILED DESCRIPTIONS OF THE EMBODIMENTS

The following preferred specific embodiments are, therefore, to be construed as merely illustrative, and non-limiting to the remainder of the disclosure in any way whatsoever. In this regard, no attempt is made to show structural details of the present invention in more detail than is necessary for fundamental understanding of the present invention; the description taken with the drawings making apparent to those skilled in the art how several forms of the present invention may be embodied in practice.

### Method of Manufacturing Bipyridinium Compound

The first aspect of the method of manufacturing a bipyridinium compound of the present invention is a method (referred to as “manufacturing method A” hereinafter) of manufacturing a bipyridinium compound denoted by general formula (A). The second aspect is a method (referred to as “manufacturing method B” hereinafter) of manufacturing a bipyridinium compound denoted by general formula (B).

Manufacturing methods A and B will be successively described below.

### Manufacturing Method A

The target product of manufacturing method A is a bipyridinium compound denoted by general formula (A) below.

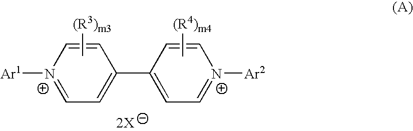

General formula (A) will be described in detail below.

Ar¹ and Ar² each independently denote an optionally substituted (hetero)aryl group.

In the present invention, the term “(hetero)aryl group” means a cyclic residue having aromatic properties, including aryl groups comprised of just carbon atoms, and heteroaryl groups comprising one or more hetero atoms such as nitrogen atoms (N), oxygen atoms (O), sulfur atoms (S), and selenium atoms (Se).

The aryl group comprised of just carbon atoms is an aryl group preferably comprising 6 to 30 carbon atoms, more preferably 6 to 20 carbon atoms, and further preferably, 6 to 16 carbon atoms, examples of which are phenyl groups, p-methylphenyl groups, naphthyl groups, and anthranyl groups.

The heteroaryl group comprising one or more hetero atoms such as N, O, S, and Se is a heteroaryl group preferably comprising 1 to 30 carbon atoms, more preferably 1 to 20 carbon atoms, and further preferably, 1 to 12 carbon atoms. Examples of the hetero atoms contained in the heteroaryl group are nitrogen atoms, oxygen atoms, and sulfur atoms. Specific examples of the heteroaryl group are pyrrole groups, pyrazole groups, imidazole groups, pyridine groups, furan groups, thiophene groups, oxazole groups, thiazole groups, and condensed ring products thereof with benzo and/or hetero rings.

The (hetero)aryl groups denoted by Ar¹ and Ar² are preferably phenyl groups, 1-naphthyl groups, or 2-naphthyl groups, more preferably phenyl groups.

The above-described (hetero)aryl group is optionally substituted. In the present invention, when the term “optionally substituted” is used for certain functional groups (such as (hetero)aryl groups, aryloxy groups, alkyl groups and the like), the type and number of substituents is not specifically limited, and when multiple substituents are present, they may be identical or different.

Examples of substituents that may be present are: alkyl groups (preferably having 1 to 30 carbon atoms, more preferably having 1 to 20 carbon atoms, and further preferably having 1 to 10 carbon atoms, such as methyl groups, ethyl groups, isopropyl groups, tert-butyl groups, n-octyl groups, n-decyl groups, n-hexadecyl groups, cyclopropyl groups, cyclopentyl groups, and cyclohexyl groups); alkenyl groups (preferably having 2 to 30 carbon atoms, more preferably having 2 to 20 carbon atoms, and further preferably having 2 to 10 carbon atoms, such as vinyl groups, allyl groups, 2-butenyl groups, and 3-pentenyl groups); alkynyl groups (preferably having 2 to 30 carbon atoms, more preferably having 2 to 20 carbon atoms, and further preferably, having 2 to 10 carbon atoms, such as propargyl groups and 3-pentynyl groups); aryl groups (preferably having 6 to 30 carbon atoms, more preferably having 6 to 20 carbon atoms, and further preferably, having 6 to 12 carbon atoms, such as phenyl groups, p-methylphenyl groups, naphthyl groups, and anthranyl groups); amino groups (preferably having 0 to 30 carbon atoms, more preferably having 0 to 20 carbon atoms, and further preferably, having 0 to 10 carbon atoms, such as amino groups, methylamino groups, dimethylamino groups, diethylamino groups, dibenzylamino groups, diphenylamino groups, and ditolylamino groups); alkoxy groups (preferably having 1 to 30 carbon atoms, more preferably having 1 to 20 carbon atoms, and further preferably, having 1 to 10 carbon atoms, such as methoxy groups, ethoxy groups, butoxy groups, 2-ethylhexyloxy groups); aryloxy groups (preferably having 6 to 30 carbon atoms, more preferably having 6 to 20 carbon atoms, and further preferably, having 6 to 12 carbon atoms, such as phenyloxy groups, 1-naphthyloxy groups, and 2-naphthyloxy groups); aromatic heterocyclic oxy groups (preferably having 1 to 30 carbon atoms, more preferably having 1 to 20 carbon atoms, and further preferably, having 1 to 12 carbon atoms, such as pyridyloxy groups, pyrazyloxy groups, pyrimidyloxy groups, and xylyloxy groups); acyl groups (preferably having 1 to 30 carbon atoms, more preferably having 1 to 20 carbon atoms, and further preferably, having 1 to 12 carbon atoms, such as acetyl groups, benzoyl groups, formyl groups, and pivaloyl groups); alkoxycarbonyl groups (preferably having 2 to 30 carbon atoms, more preferably having 2 to 20 carbon atoms, and further preferably, having 2 to 12 carbon atoms, such as methoxycarbonyl groups and ethoxycarbonyl groups); aryloxycarbonyl groups (preferably having 7 to 30 carbon atoms, more preferably having 7 to 20 carbon atoms, and further preferably, having 7 to 12 carbon atoms, such as phenyloxycarbonyl groups); acyloxy groups (preferably having 2 to 30 carbon atoms, more preferably having 2 to 20 carbon atoms, and further preferably, having 2 to 10 carbon atoms, such as acetoxy groups and benzoyloxy groups); acylamino groups (preferably having 2 to 30 carbon atoms, more preferably having 2 to 20 carbon atoms, and further preferably, having 2 to 10 carbon atoms, such as acetylamino groups and benzoylamino groups); alkoxycarbonylamino groups (preferably having 2 to 30 carbon atoms, more preferably having 2 to 20 carbon atoms, and further preferably, having 2 to 12 carbon atoms, such as methoxycarbonylamino groups); aryloxycarbonylamino groups (preferably having 7 to 30 carbon atoms, more preferably having 7 to 20 carbon atoms, and further preferably, having 7 to 12 carbon atoms, such as phenyloxycarbonylamino groups); sulfonylamino groups (preferably having 1 to 30 carbon atoms, more preferably having 1 to 20 carbon atoms, and further preferably, having 1 to 12 carbon atoms, such as methanesulfonylamino groups and benzenesulfonylamino groups); sulfamoyl groups (preferably having 0 to 30 carbon atoms, more preferably having 0 to 20 carbon atoms, and further preferably, having 0 to 12 carbon atoms, such as sulfamoyl groups, methylsulfamoyl groups, dimethylsulfamoyl groups, and phenylsulfamoyl groups); carbamoyl groups (preferably having 1 to 30 carbon atoms, more preferably having 1 to 20 carbon atoms, and further preferably, having 1 to 12 carbon atoms, such as carbamoyl groups, methylcarbamoyl groups, diethylcarbamoyl groups, and phenylcarbamoyl groups); alkylthio groups (preferably having 1 to 30 carbon atoms, more preferably having 1 to 20 carbon atoms, and further preferably, having 1 to 12 carbon atoms, such as methylthio groups and ethylthio groups); arylthio groups (preferably having 6 to 30 carbon atoms, more preferably having 6 to 20 carbon atoms, and further preferably, having 6 to 12 carbon atoms, such as phenylthio groups); aromatic heterocyclic thio groups (preferably having 1 to 30 carbon atoms, more preferably having 1 to 20 carbon atoms, and further preferably, having 1 to 12 carbon atoms, such as pyridylthio groups, 2-benzimidazolylthio groups, 2-benzoxazolylthio groups, and 2-benzthiazolylthio groups); sulfonyl groups (preferably having 1 to 30 carbon atoms, more preferably having 1 to 20 carbon atoms, and further preferably, having 1 to 12 carbon atoms, such as mesyl groups and tosyl groups); sulfinyl groups (preferably having 1 to 30 carbon atoms, more preferably having 1 to 20 carbon atoms, and further preferably, having 1 to 12 carbon atoms, such as methanesulfinyl groups and benzenesulfinyl groups); ureido groups (preferably having 1 to 30 carbon atoms, more preferably having 1 to 20 carbon atoms, and further preferably, having 1 to 12 carbon atoms, such as ureido groups, methylureido groups, and phenylureido groups); phosphoramide groups (preferably having 1 to 30 carbon atoms, more preferably having 1 to 20 carbon atoms, and further preferably, having 1 to 12 carbon atoms, such as diethyl phosphoramide groups and phenyl phosphoramide groups); hydroxy groups; mercapto groups; halogen atoms (such as fluorine atoms, chlorine atoms, bromine atoms, and iodine atoms); cyano groups; sulfo groups; carboxyl groups; nitro groups; hydroxamic acid groups; sulfino groups; hydrazino groups; imino groups; aromatic heterocyclic groups (preferably having 1 to 30 carbon atoms, more preferably having 1 to 12 carbon atoms, with one or more hetero atoms such as nitrogen atoms, oxygen atoms, and/or sulfur atoms, specific examples of which being imidazolyl groups, pyridyl groups, quinolyl groups, furyl groups, thienyl groups, piperidyl groups, morpholino groups, benzoxazolyl groups, benzimidazolyl groups, benzthiazolyl groups, carbazolyl groups, and azepinyl groups); and silyl groups (preferably having 3 to 40 carbon atoms, more preferably having 3 to 30 carbon atoms, and further preferably, having 3 to 24 carbon atoms, such as trimethylsilyl groups and triphenylsilyl groups). These substituents may be further substituted.

The above-listed substituents are examples of the substituents that may be present on the (hetero)aryl groups denoted by Ar¹ and Ar². Preferable substituents are: alkyl groups, aryl groups, amino groups, alkoxy groups, aryloxy groups, aromatic heterocyclic oxy groups, acyl groups, acyloxy groups, acylamino groups, carbamoyl groups, sulfonylamino groups, sulfamoyl groups, ureido groups, hydroxy groups, cyano groups, halogen atoms, and aromatic heterocyclic groups; more preferable groups are alkyl groups, aryl groups, alkoxy groups, aryloxy groups, acyl groups, acyloxy groups, acylamino groups, carbamoyl groups, ureido groups, hydroxy groups, and aromatic heterocyclic groups.

In general formula (A), X denotes a halogen atom or RSO₃, and R denotes an optionally substituted aryl group or alkyl group. In the present invention, a “halogen atom” may be any one from among a fluorine atom, chlorine atom, bromine atom, or iodine atom. A chlorine atom or bromine atom is preferable from the perspectives of the ease of procuring starting materials and cost, with a chlorine atom being preferred. Aryl groups and alkyl groups denoted by R are as set forth for the aryl groups and alkyl groups serving as possible substituents on the (hetero)aryl groups denoted by Ar¹ and Ar² above. The aryl group and alkyl group denoted by R are optionally substituted. Examples of the substituents are those given by way of example for possible substituents on the (hetero)aryl groups denoted by Ar¹ and Ar² above. Of these, halogen atoms are preferred as substituents. Specific examples of RSO₃ are CH₃SO₃, CF₃SO₃, CF₃(CF₂)₇SO₃, and p-toluenesulfonyl groups, with CH₃SO₃ and p-toluenesulfonyl groups being preferable.

In general formula (A), R³ and R⁴ each independently denote a substituent. Examples of the substituents denoted by R³ and R⁴ are those set forth above. Preferable examples are alkyl groups, amino groups, alkoxy groups, acyl groups, acyloxy groups, acylamino groups, carbamoyl groups, cyano groups, and halogen atoms. More preferable examples are alkyl groups and halogen atoms. Each of R³ and R⁴ may form a ring with a pyridine ring to which it substitutes.

m3 and m4 each independently denote an integer ranging from 0 to 4. m3 and m4 each preferably denote an integer ranging from 0 to 2, more preferably 0 or 1. When m3 denotes an integer ranging from 2 to 4, plural R³ present may be identical or different from each other and when m4 denotes an integer ranging from 2 to 4, plural R⁴ present may be identical or different from each other.

In manufacturing method A, the bipyridinium compound denoted by general formula (A) is manufactured in steps (a) to (d) below.

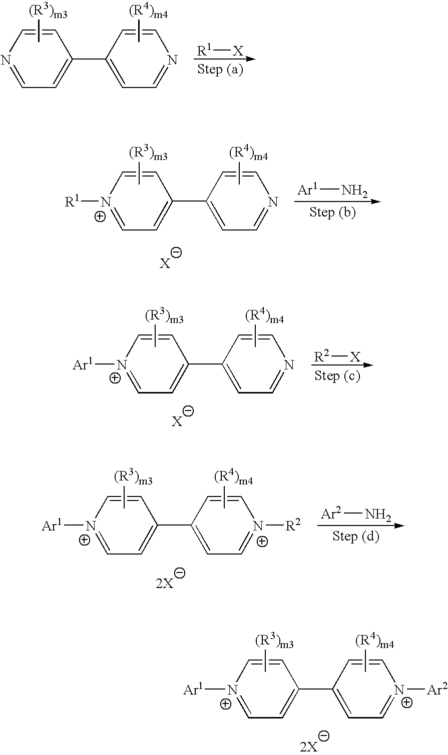

Each of the above steps will be described below.

### Step (a)

In step (a), a bipyridine compound denoted by general formula (1):

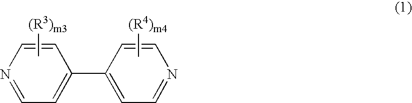

is reacted with a (hetero)arylhalogen compound denoted by general formula (2):

R¹—X (2)

in a solvent to manufacture an N-((hetero)aryl)pyridinium compound denoted by general formula (3).

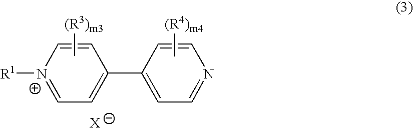

In general formula (1), R³, R⁴, m3 and m4 are respectively defined as in general formula (A). The details thereof are as set forth above.

In general formula (2), R¹ denotes an optionally substituted (hetero)aryl group.

When R¹ denotes an aryl group comprised of just carbon atoms, examples of the aryl group are phenyl groups and naphthyl groups. Of these, phenyl groups are preferable.

When R¹ denotes an aryl group comprised of just carbon atoms having a substituent, the aryl group is preferably a phenyl group with an electron-withdrawing group substituted on the ring thereof. Examples of the electron-withdrawing are cyano groups, nitro groups, acyl groups having 1 to 6 carbon atoms, alkoxycarbonyl groups having 1 to 6 carbon atoms, and alkylsulfonyl groups having 1 to 6 carbon atoms. Of these, cyano groups, nitro groups, and alkylsulfonyl groups having 1 to 6 carbon atoms are preferable. Nitro groups are the substituent of greatest preference. A 2,4-dinitrophenyl group is the group of greatest preference for the aryl group comprised of just carbon atoms denoted by R¹.

When R¹ denotes a heteroaryl group comprising a hetero atom such as N, O, S, or Se, the heteroaryl group preferably has 1 to 20 carbon atoms in the ring structure thereof, with 3 to 10 carbon atoms being preferred. Examples of the heteroaryl group are: oxazole rings, benzoxazole rings, thiazole rings, benzothiazole rings, imidazole rings, benzimidazole rings, pyridine rings, and pyrimidine rings. Of these, benzoxazole rings, thiazole rings, benzothiazole rings, imidazole rings, benzoimidazole rings, and pyrimidine rings are preferred, and thiazole rings, benzothiazole rings, and pyrimidine rings are of greater preference.

When R¹ denotes a heteroaryl group, it may comprise a substituent. The details of the substituent are as set forth above.

In general formula (2), X is defined as in general formula (A). The details thereof are as set forth above.

In general formula (3), R¹, R³, R⁴, m3, m4 and X are respectively defined as in general formula (A) or (2). The details thereof are as set forth above.

In step (a), the 4,4′-bipyridine denoted by general formula (1) is reacted with the (hetero)arylhalogen compound denoted by general formula (2) in a solvent. The concentration of the 4,4′-bipyridine denoted by general formula (1) in the reaction solution is, for example, 5 to 60 weight percent, preferably 10 to 50 weight percent. The (hetero)arylhalogen compound denoted by general formula (2) is preferably employed in a quantity falling within a range of 0.5 to 1.0 moles, more preferably a range of 0.6 to 0.9 moles, and further preferably, a range of 0.6 to 0.8 moles, per mol of 4,4′-bipyridine denoted by general formula (1). Use of the (hetero)arylhalogen compound in excessive amount is undesirable on an industrial-scale manufacturing because it complicates subsequent processing, increases the amount of waste product, and increases the cost.

By way of example, the reaction of the 4,4′-bipyridine denoted by general formula (1) and the (hetero)arylhalogen compound denoted by general formula (2) can be conducted at a reaction temperature ranging from 10 to 180° C., preferably ranging from 60 to 150° C., and more preferably ranging from 70 to 140° C. The reaction time varies with the charge amount and reaction temperature, but is normally equal to or less than 9 hours. It is about 2 to 8 hours, for example. An inert gas is not required while implementing the reaction, but the reaction can be conducted under an argon or nitrogen gas flow.

The bipyridinium compound denoted by general formula (3) can be obtained in step (a). The progress of the reaction can be confirmed by liquid chromatography, NMR, or the like.

In manufacturing method A, once step (a) has been completed, the reaction solution can be used in subsequent steps without conducting the step of separating the bipyridinium compound denoted by general formula (3). When a separation step is conducted, a known purification method, such as crystallization, can be employed.

### Step (b)

In step (b), the bipyridinium compound denoted by general formula (3):

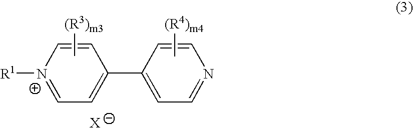

that has been obtained in step (a) is reacted with an amine compound denoted by general formula (4):

Ar¹—NH₂ (4)

in a solvent to manufacture an N-aryl-substituted bipyridinium compound denoted by general formula (5).

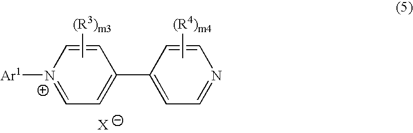

In general formula (4), Ar¹ is defmed as in general formula (A), and the details thereof are as set forth above.

In general formula (5), Ar¹, R³, R⁴, m3, m4 and X are respectively defined as in general formula (A). The details thereof are as set forth above.

In step (b), the bipyridinium compound denoted by general formula (3) is reacted with the amine compound denoted by general formula (4) in a solvent. As stated above, with the completion of step (a), the amine compound denoted by general formula (4) can be added to the reaction solution to conduct the reaction without conducting a separation step.

The concentration of the bipyridinium compound denoted by general formula (3) in the reaction solution is, for example, 5 to 50 weight percent, preferably 10 to 40 weight percent. The amine compound denoted by general formula (4) is employed in a quantity, for example, ranging from 0.5 to 1.0 mole, preferably ranging from 0.6 to 0.9 mole, and more preferably ranging from 0.6 to 0.8 mole, per mole of bipyridinium compound denoted by general formula (3). By way of example, the reaction can be implemented within a range of 10 to 180° C., preferably a range of 60 to 150° C., and more preferably, a range of 70 to 140° C. The reaction time varies with the charge amount and reaction temperature, but is normally equal to or less than 9 hours. It is about 2 to 8 hours, for example. An inert gas is not required while implementing the reaction, but the reaction can be conducted under an argon or nitrogen gas flow.

The N-aryl-substituted bipyridinium compound denoted by general formula (5) can be obtained in step (b). The progress of the reaction can be confirmed by liquid chromatography, NMR, or the like.

Solvents such as a ketone solvents such as acetone and cyclohexanone; amide solvents such as N-methylpyrrolidone, N,N-dimethylformamide, and N,N-dimethylacetamide; alcohol solvents such as methanol, ethanol, propanol, butanol, ethylene glycol, propylene glycol, butylene glycol, glycerin, diethylene glycol, and triethylene glycol; and solvents capable of mixing readily with water, such as dimethylsulfoxide and acetonitrile, can be employed singly, or mixed together for use, in steps (a) and (b). Acetonitrile, amide solvents, and alcohol solvents are preferably employed.

Extraction with water is preferably conducted after conducting steps (a) and (b) sequentially in manufacturing method A. Such extraction with water permits the obtaining of the N-aryl-substituted bipyridinium compound denoted by general formula (5) with good yield and purity. It is not clearly known why the use of a reaction solvent in the form of acetonitrile, an amide, or an alcohol solvent, as set forth above, is effective. However, we presume the reason to be as follows. In the reaction between the (hetero)arylhalogen compound and 4,4′-bipyridine, two (hetero)arylhalogen compounds react with the 4,4′-bipyridinine to form a product. This product produces impurities by reaction with amine compounds. These impurities are thought to dissolve in water and solvents of high polarity, remaining in solution when the product is removed, and thereby permitting control of precipitation of just the product.

### Step (c)

In step (c), the N-aryl-substituted bipyridinium compound denoted by general formula (5):

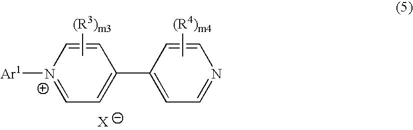

that has been obtained in step (b) is reacted in solvent with a (hetero)arylhalogen compound denoted by general formula (6):

R²—X (6)

to manufacture a bipyridinium compound denoted by general formula (7).

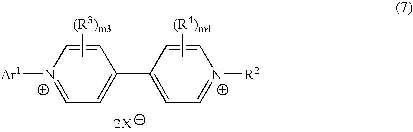

In general formula (6), R² denotes an optionally substituted (hetero)aryl group. The details thereof are identical to those set forth for R¹ in general formula (2) above.

In general formula (6), X is defined as in general formula (A), and the details thereof are as set forth above.

In general formula (7), Ar¹, R², R³, R⁴, m3, m4 and X are respectively defined as in general formula (A) or (6). The details thereof are as set forth above.

In step (c), the N-aryl-substituted bipyridinium compound denoted by general formula (5) is reacted with the (hetero)arylhalogen compound denoted by general formula (6) in a solvent. The concentration of the N-aryl-substituted bipyridinum compound denoted by general formula (5) in the reaction solvent is, for example, 5 to 50 weight percent, preferably 10 to 40 weight percent. The (hetero)arylhalogen compound denoted by general formula (6) is employed in a quantity, for example, ranging from 2.0 to 5.0 moles, preferably ranging from 2.0 to 4.0 moles, more preferably ranging from 2.5 to 3.5 moles, per mole of the N-aryl-substituted bipyridinium compound denoted by general formula (5). Use of the (hetero)arylhalogen compound in excessive amount is undesirable on an industrial-scale manufacturing because it complicates subsequent processing operations, increases the amount of waste product, and increases cost.

By way of example, the reaction between the N-aryl-substituted bipyridinium compound denoted by general formula (5) and the (hetero)arylhalogen compound denoted by general formula (6) can be conducted within a range of 10 to 180° C., preferably within a range of 60 to 150° C., and more preferably, within a range of 70 to 140° C. The reaction time varies with the charge amount and reaction temperature, but is normally equal to or less than 9 hours. It is about 2 to 8 hours, for example. An inert gas is not required while implementing the reaction, but the reaction can be conducted under an argon or nitrogen gas flow.

The bipyridinium compound denoted by general formula (7) can be obtained in step (c). The progress of the reaction can be confirmed by liquid chromatography, NMR, or the like.

In manufacturing method A, once step (c) has been completed, the reaction solution can be used in subsequent steps without conducting the step of separating the bipyridinium compound denoted by general formula (7). When a separation step is conducted, a known purification method, such as crystallization, can be employed.

### Step (d)

In step (d), the bipyridinium compound denoted by general formula (7):

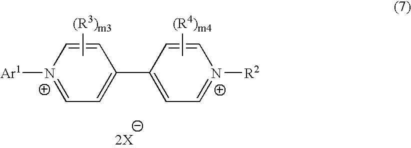

that has been obtained in step (c) is reacted in a solvent with the amine compound denoted by general formula (8):

Ar²—NH₂ (8)

to manufacture the bipyridinium compound denoted by general formula (A).

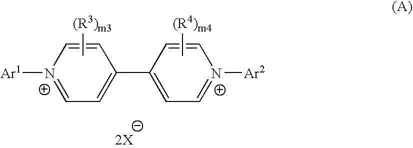

In general formula (8), Ar² is defined as in general formula (A), and the details thereof are as set forth above.

In step (d), the bipyridinium compound denoted by general formula (7) is reacted with the amine compound denoted by general formula (8) in a solvent. The reaction can be conducted by adding the amine compound denoted by general formula (8) to the reaction solution without conducting a separation step following the conclusion of step (c), as set forth above.

The concentration of the bipyridinium compound denoted by general formula (7) in the reaction solution is, for example, 5 to 50 weight percent, preferably 10 to 40 weight percent. The amine compound denoted by general formula (8) is employed in a quantity, for example, ranging from 0.5 to 2.0 moles, preferably 0.8 to 1.5 moles, more preferably 0.9 to 1.2 moles, per mole of the bipyridinium compound denoted by general formula (7). By way of example, the reaction can be conducted within a range of 10 to 180° C., preferably 60 to 150° C., and more preferably 70 to 140° C. The reaction time varies with the charge amount and reaction temperature, but is normally equal to or less than 9 hours. It is about 2 to 8 hours, for example. An inert gas is not required while implementing the reaction, but the reaction can be conducted under an argon or nitrogen gas flow.

With the conclusion of step (d), extraction with water can be conducted to obtain the targeted bipyridinium compound denoted by general formula (A) at good yield and purity.

Examples of the solvent employed in steps (c) and (d) are the solvents given by way of example for use in steps (a) and (b) above. The use of a solvent in the form of acetonitrile, an amide, or an alcohol is effective for conducting steps (c) and (d) successively. Further, in steps (c) and (d), among the above solvents, it is effective to employ a solvent with a high boiling point (such as a boiling point of 80 to 200° C.), such as dimethylformamide, dimethylacetamide, N-methylpyrrolidone, dimethylsulfoxide, glycerol, ethylene glycol, ethylene glycol monoethyl ether, and diethylene glycol diethyl ether. Although the reason is unclear, we presume that a solvent with a high boiling point permits raising of the reaction temperature, facilitating dissolution of the bipyridinium compound in the water employed during extraction and the like, thereby activating the reaction and affecting solubility.

As an alternative to, or in addition to, extraction with water as set forth above, the target compound can be separated from the reaction mixture following the conclusion of the reaction by using the usual separation and purification means. For example, adding a poor solvent to the reaction mixture and then cooling it causes the target compound to crystallize and precipitate out. The precipitate can then be separated by the usual separation methods based on solid-liquid separation or the like.

The 4,4′-bipyridinium compound denoted by general formula (A) that has been obtained as set forth above is normally of high enough purity for use without further purification. However, further purification may be conducted based on the application and objective. Methods commonly employed to purify organic compounds, such as slurry suspension purification and recrystallization employing an organic solvent such as methanol, ethanol, 2-propylalcohol, acetone, acetonitrile, methyl ethyl ketone, N,N-dimethylformamide, and N,N-dimethylacetamide, can be applied as the purification method. Production of the target compound can be confirmed by a known method such as NMR, mass spectrometry, or elemental analysis.

Manufacturing method A as set forth above permits a reduction in the frequency of separation of intermediates, essentially reducing four steps to 2 steps. It is thus a good industrial manufacturing method in terms of efficiency, cost, and safety. The bipyridinium compound denoted by general formula (A) that is the target compound can also be obtained as a corresponding salt. The anion forming a salt with the bipyridinium compound denoted by general formula (A) can be either an organic or inorganic anion. Examples are halide ions (Cl⁻, Br⁻, I⁻, and the like), sulfonate ions (CH₃SO₃⁻, CF₃SO₃⁻, CF₃(CF₂)₇SO₃⁻, p-toluene sulfonate ions, naphthalene-1,5-disulfonate ions, and the like), sulfuric acid ions (CH₃SO₄⁻ and the like), ClO₄⁻, BF₄⁻, SbF₆⁻, phosphoric acid ions (PF⁶⁻,

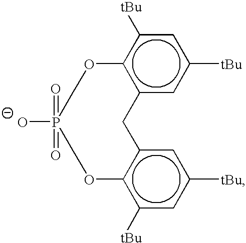

and the like), and metal complex ions (for example, the following anions).

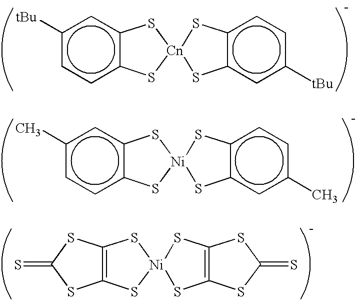

Preferable anions are Cl⁻, Br⁻, I⁻, p-toluene sulfonate ions, ClO₄⁻, BF₄⁻, and SbF₆⁻.

In general formula (A), Ar¹ and Ar² may be identical or different. It is possible to obtain asymmetric 4,4′-bipyridinium compounds and salts thereof in which Ar¹ and Ar² are different by manufacturing method A. The details of the salts are as set forth above.

The asymmetric 4,4′-bipiridinium compound is preferably denoted by general formula (C) below.

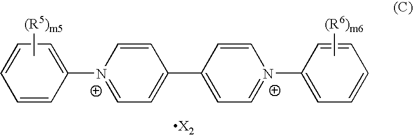

In general formula (C), R⁵ and R⁶ each independently denote a substituent that may form a ring with a benzene ring to which the substituent substitutes. Preferable examples of the substituent are the substituents exemplified for the heteroaryl group denoted by R¹ in general formula (2) above. More preferable examples are alkyl groups, alkoxy groups, aryloxy groups, acyl groups, acyloxy groups, acylamino groups, carbamoyl groups, hydroxy groups, cyano groups, halogen atoms, and heteroaryl groups. Further preferable examples are alkyl groups, alkoxy groups, aryloxy groups, acyl groups, acylamino groups, carbamoyl groups, halogen atoms, and heteroaryl groups.

m5 and m6 each independently denote an integer ranging from 0 to 5, preferably an integer ranging from 0 to 3, and more preferably 0 or 1.

When m5 denotes an integer ranging from 2 to 5, plural R⁵ present may be identical or different from each other, and when m6 denotes an integer ranging from 2 to 5, plural R⁶ present may be identical or different from each other.

In general formula (C), m5 and m6 do not both denote 0, and the substituent group denoted by (R⁵)m5 and the substituent group denoted by (R⁶)m6 are not identical.

In general formula (C), X² denotes an anion that neutralizes a charge within a molecule. An example is the anion denoted by 2X⁻ in general formula (A). The details are as set forth above.

A number of the above-described reaction substances and products are specifically described below. However, the present invention is not limited to the specific examples given below.

Specific examples of the (hetero)arylhalogen compound denoted by general formula (2)

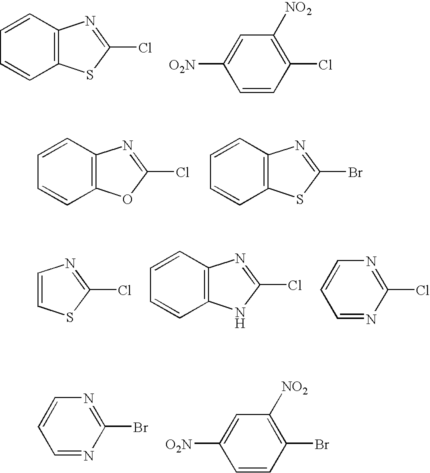

Specific examples of the bipyridinium compound denoted by general formula (3)

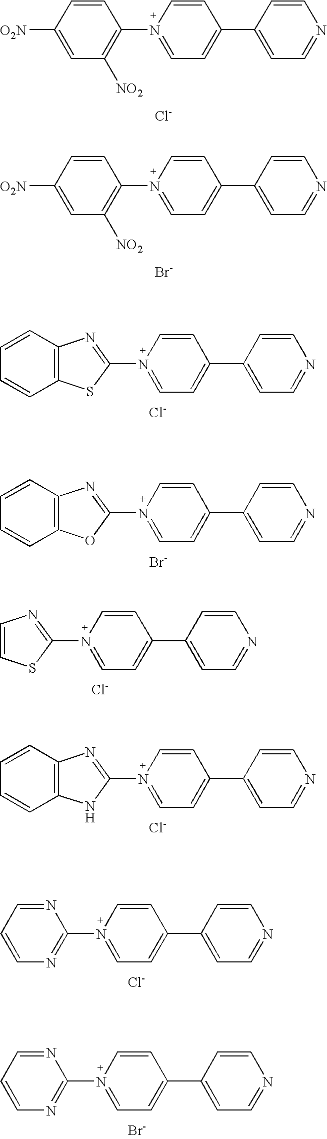

Specific examples of the amine compound denoted by general formula (4)

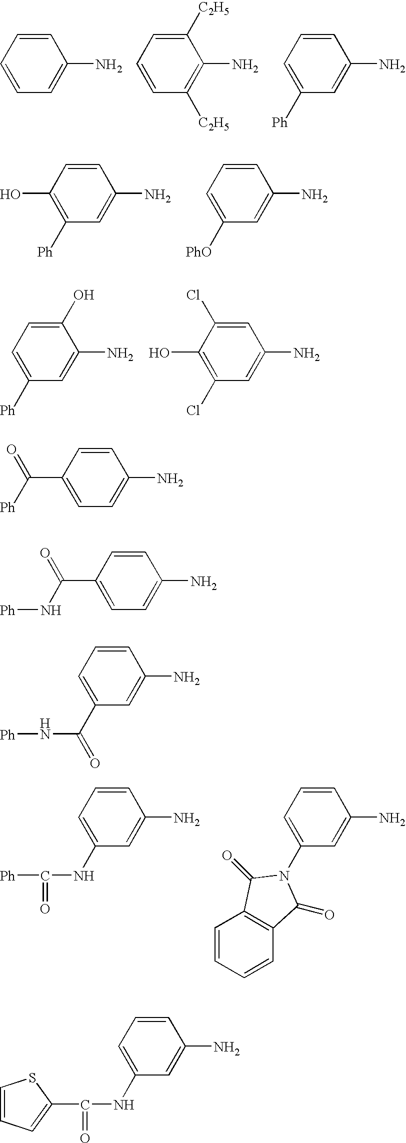

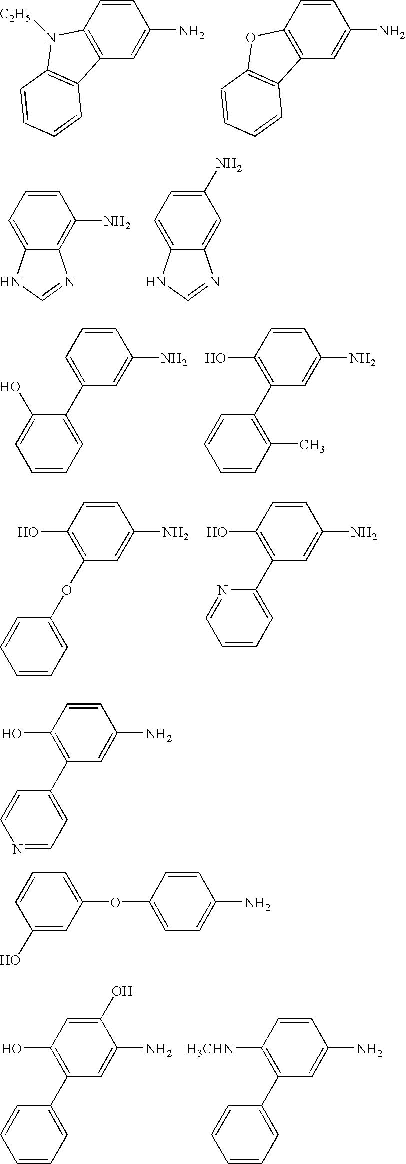

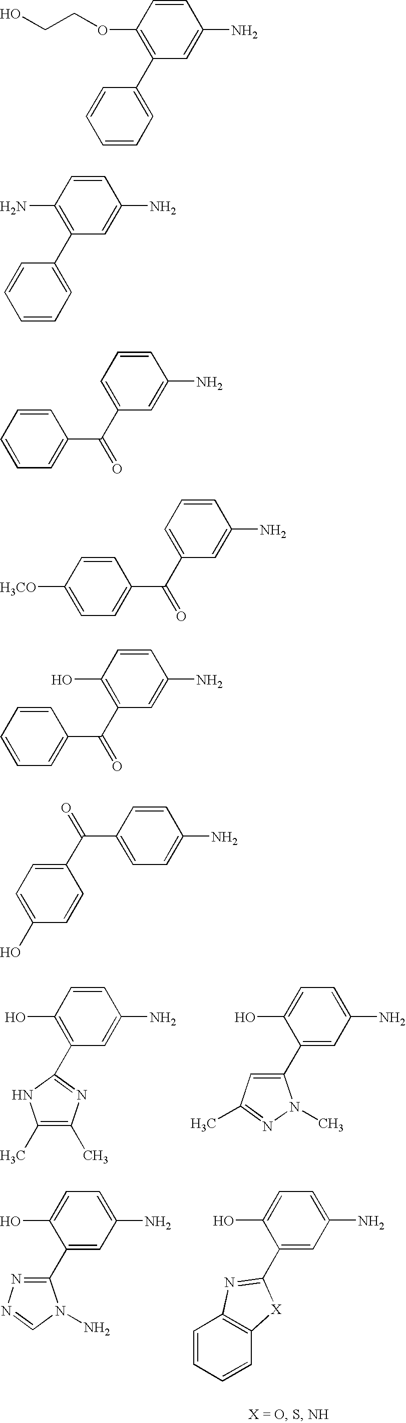

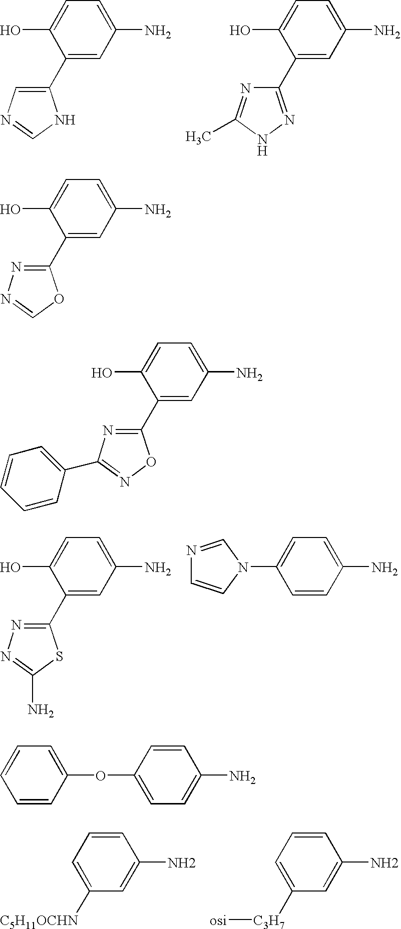

A specific example of the N-aryl-substituted bipyridinium compound denoted by general formula (5) is the product obtained by reacting the specific example compound of the amine compound denoted by general formula (4) above with aniline.

Specific examples of the (hetero)arylhalogen compound denoted by general formula (6)

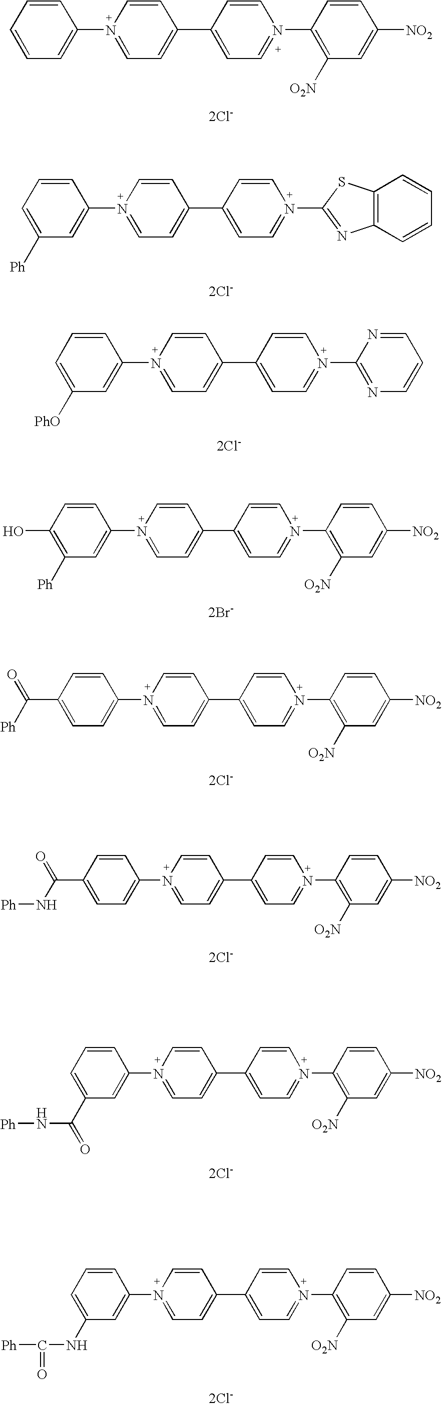

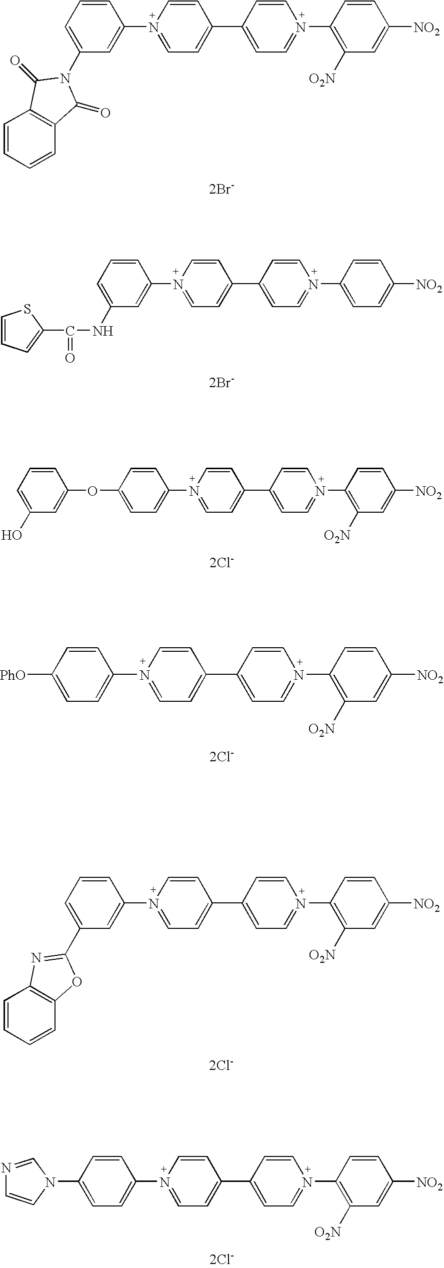

Specific examples of the cation moiety in the bipyridinium compound denoted by general formula (7), (A) or (C)

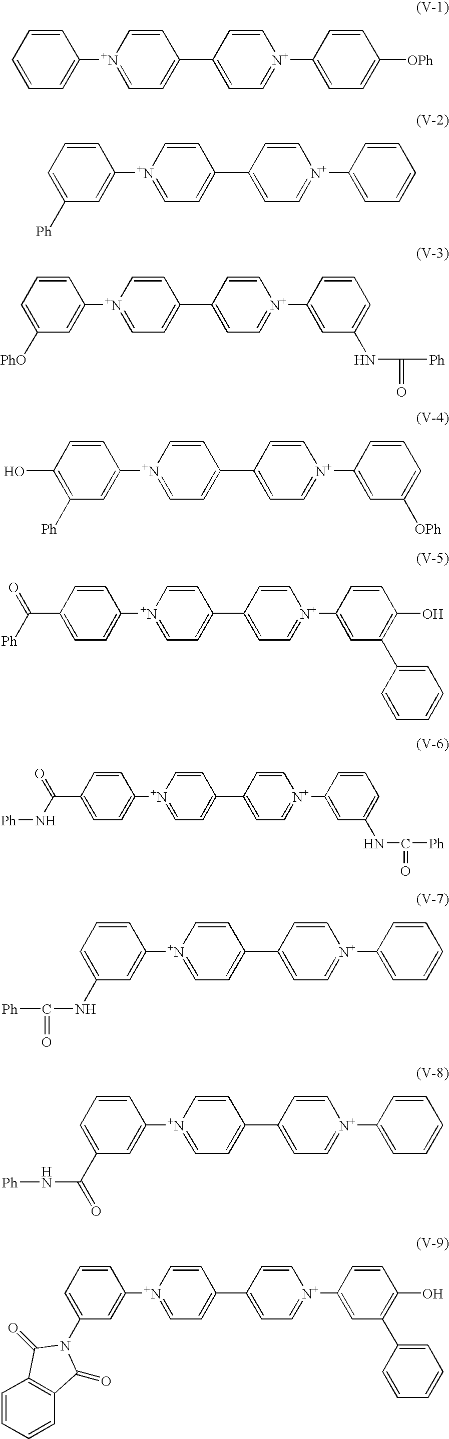

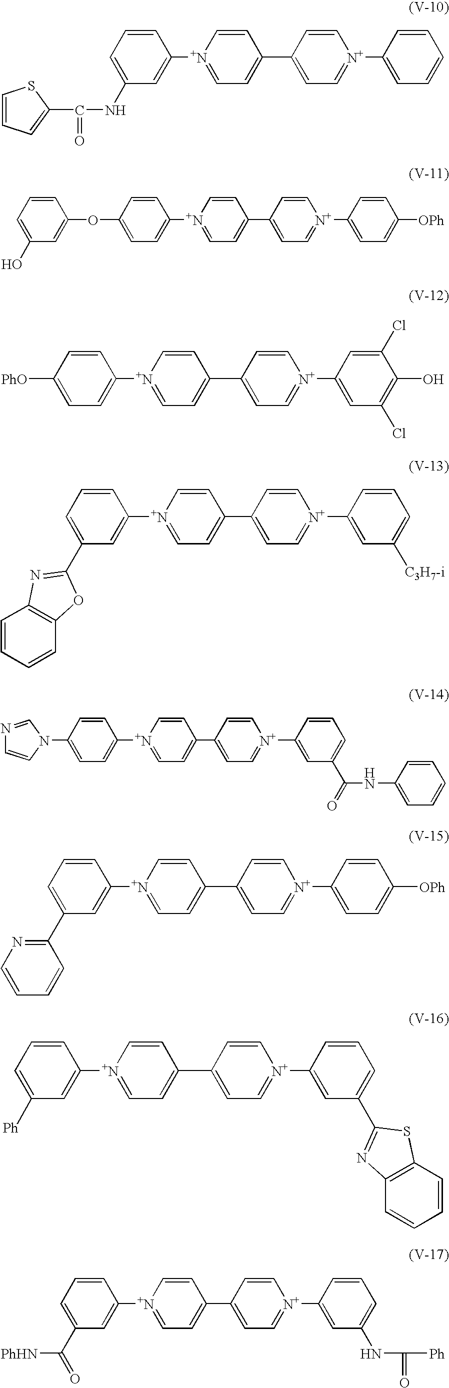

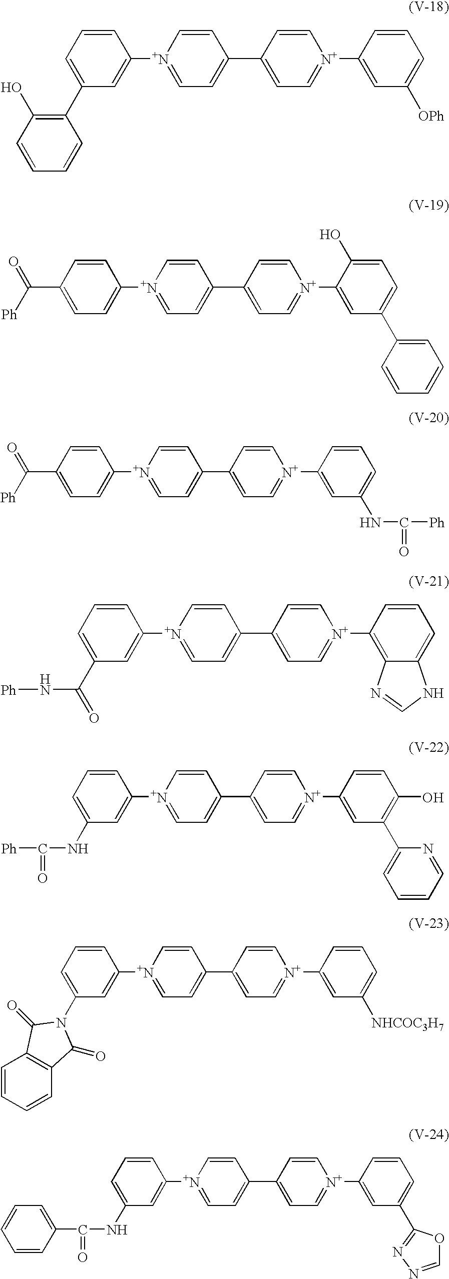

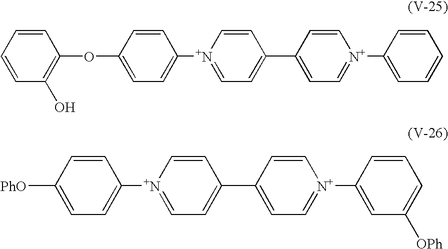

### Manufacturing Method B

Manufacturing method B will be described next.

The target product of manufacturing method B is the bipyridinium compound denoted by general formula (B) below.

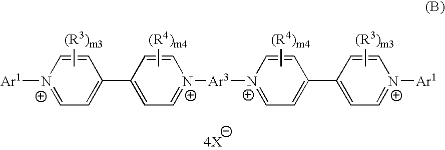

In general formula (B), Ar³ denotes an optionally substituted (hetero)arylene group. In the present invention, “(hetero)arylene group” means a cyclic residue having aromatic properties, including arylene groups comprised of just carbon atoms, and heteroarylene groups comprising on or more hetero atoms such as nitrogen atoms (N), oxygen atoms (O), sulfur atoms (S), and selenium atoms (Se).

Ar³ preferably denotes an o-phenylene group, m-phenylene group, p-phenylene group, or divalent group in which two phenyl groups are linked by a divalent linking group. The divalent linking group is preferably the following linking group.

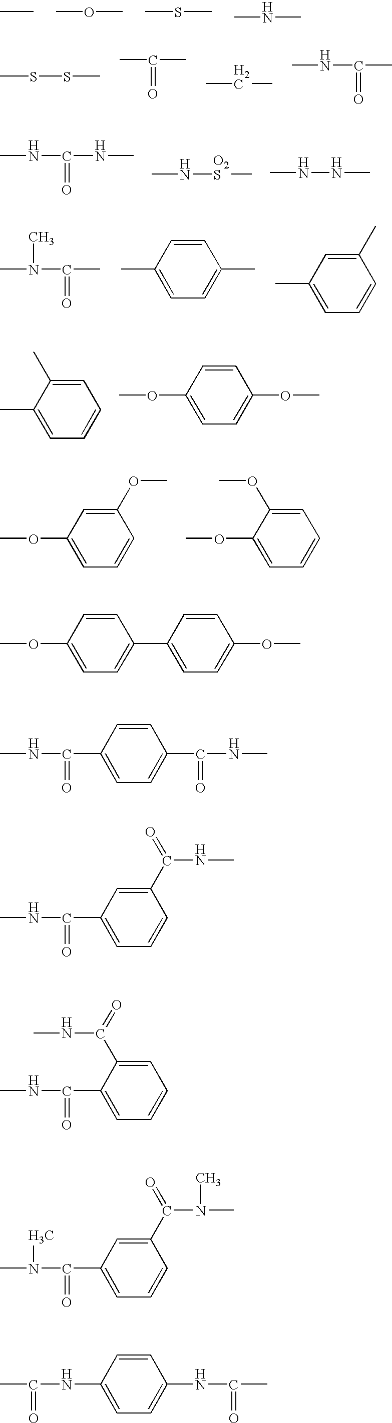

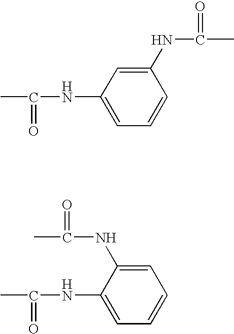

In general formula (B), Ar¹, R³, R⁴, m3, m4 and X are respectively defined as in general formula (A). The details thereof are as set forth above.

The bipyridinium compound denoted by general formula (B) above is manufactured in steps (a) to (c) and (e) below in manufacturing method B.

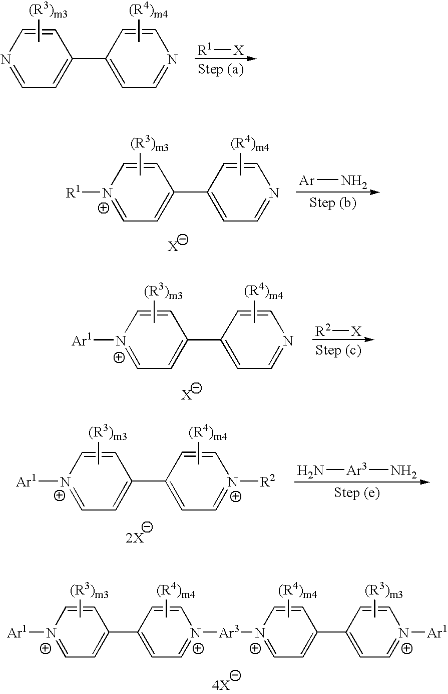

The details of steps (a), (b), and (c) in manufacturing method B are as set forth for manufacturing method A above.

Step (e) will be described below.

### Step (e)

In step (e), the bipyridinium compound denoted by general formula (7) that has been obtained in step (c) is reacted with an diamine compound denoted by general formula (9):

H₂N—Ar³—NH₂ (9)

in a solvent to manufacture a bipyridinium compound denoted by general formula (B).

In general formula (9), Ar³ is defined as in general formula (B) and the details thereof are as set forth above.

In step (e), the amine compound employed in step (d) of manufacturing method A is replaced with a diamine compound denoted by general formula (9) to obtain a bispyridinium compound denoted by general formula (B).

In step (e), in the same manner as in step (d), the diamine compound denoted by general formula (9) can be added to the reaction solution without separating the bipyridinium compound denoted by general formula (7) that has been obtained in step (c), and a reaction can be conducted. The concentration of the bipyridinium compound denoted by general formula (7) in the reaction solution is, for example, 5 to 60 weight percent, preferably 10 to 50 weight percent. The diamine compound denoted by general formula (9) is employed in a quantity, for example, ranging from 0.25 to 0.5 mole, preferably 0.3 to 0.45 mole, per mole of the bipyridinium compound denoted by general formula (7). By way of example, the reaction can be conducted within a range of 10 to 180° C., preferably within a range of 60 to 150° C., and more preferably within a range of 70 to 140° C. The reaction time varies with the charge amount and reaction temperature, but is normally equal to or less than 9 hours. It is about 2 to 8 hours, for example. An inert gas is not required while implementing the reaction, but the reaction can be conducted under an argon or nitrogen gas flow.

The remaining details relating to manufacturing method B are as set forth above for manufacturing method A. As in manufacturing method A, in manufacturing B, it is preferable that no separation step is conducted between step (a) and step (b) and between step (c) and step (e), and an extraction step with water is preferably conducted following step (b), and following step (e). This permits a reduction in the frequency of separation of intermediates, essentially reducing four steps to 2 steps. This is thus desirable from the perspectives of efficiency, cost, and safety. Production of the target compound can be confirmed by a known method, such as NMR, mass spectrometry, or elemental analysis.

Specific examples of the bipyridinium compound denoted by general formula (B) are given below. However, the present invention is not limited to the specific examples below.

Specific examples of the bipyridinium compound denoted by general formula (B)

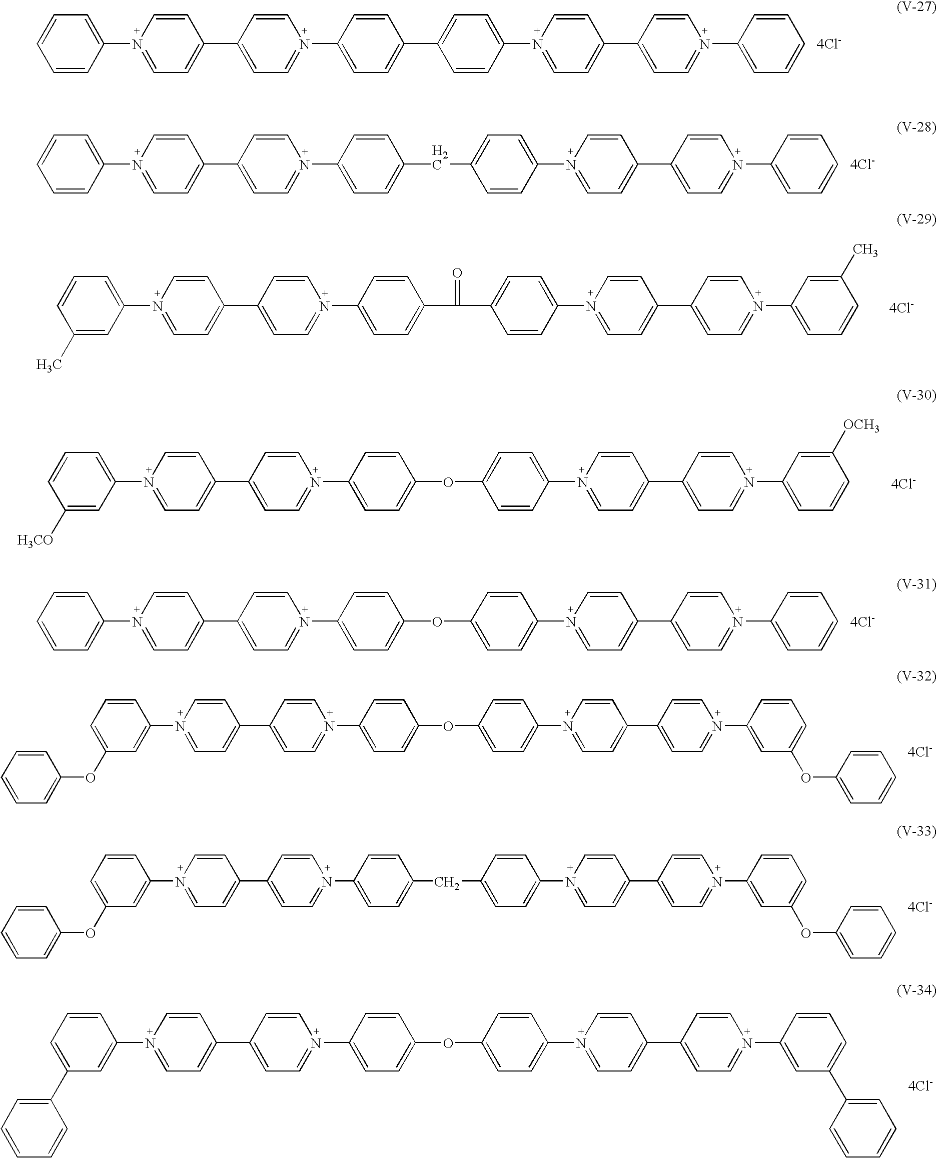

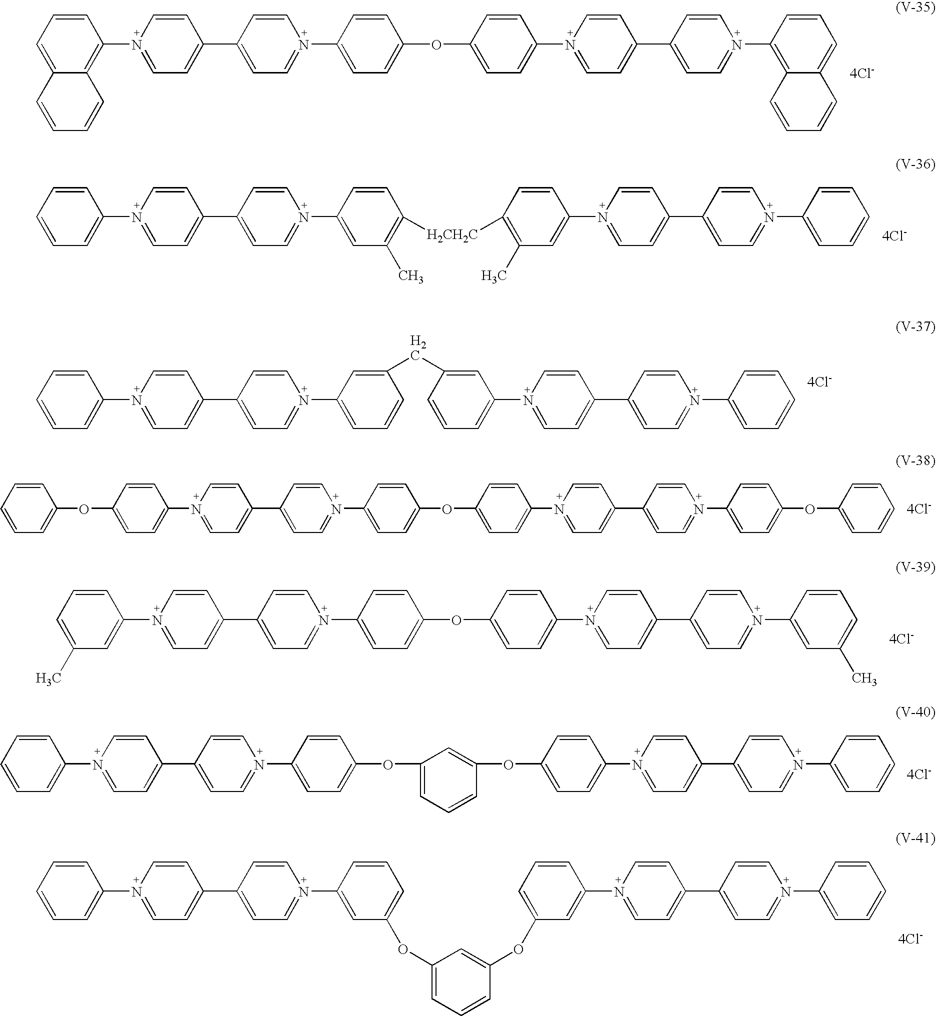

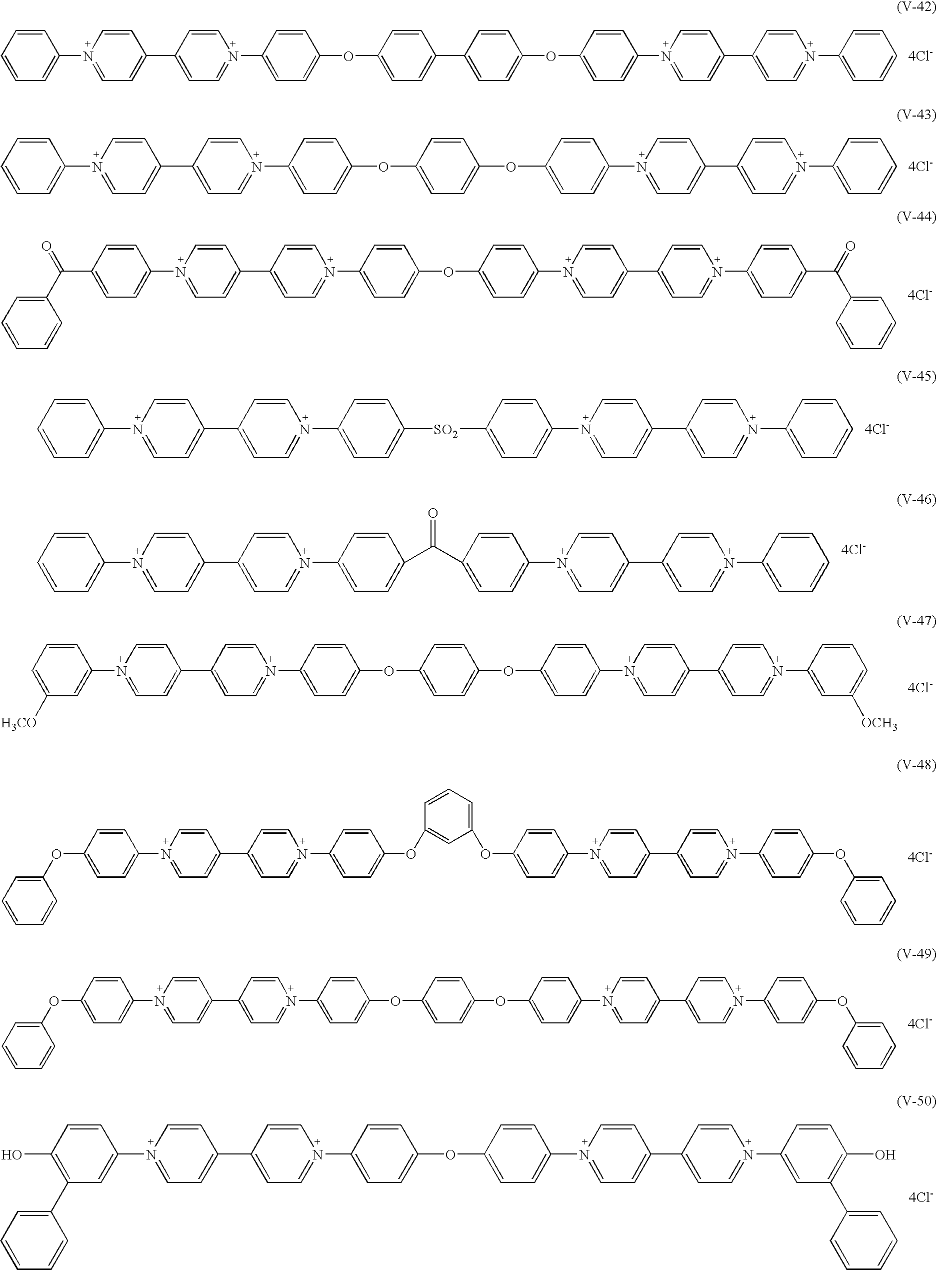

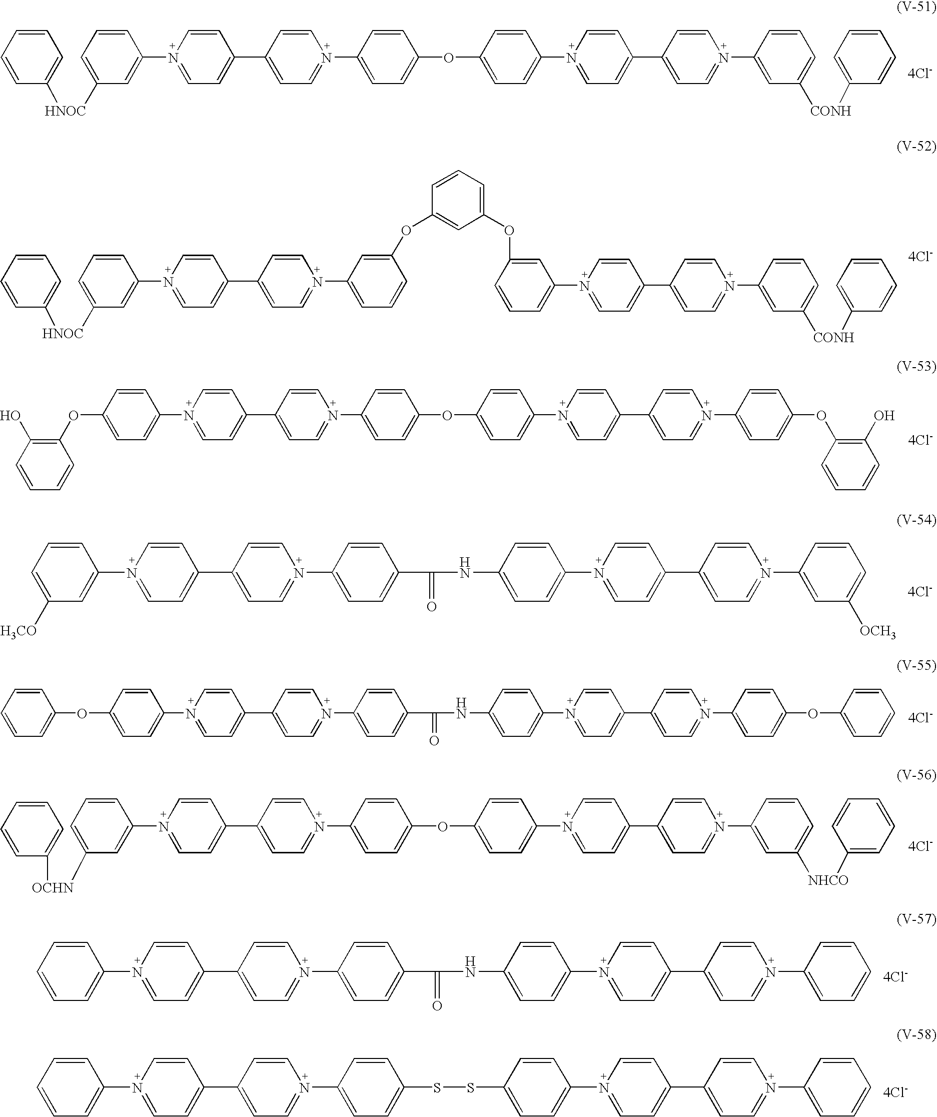

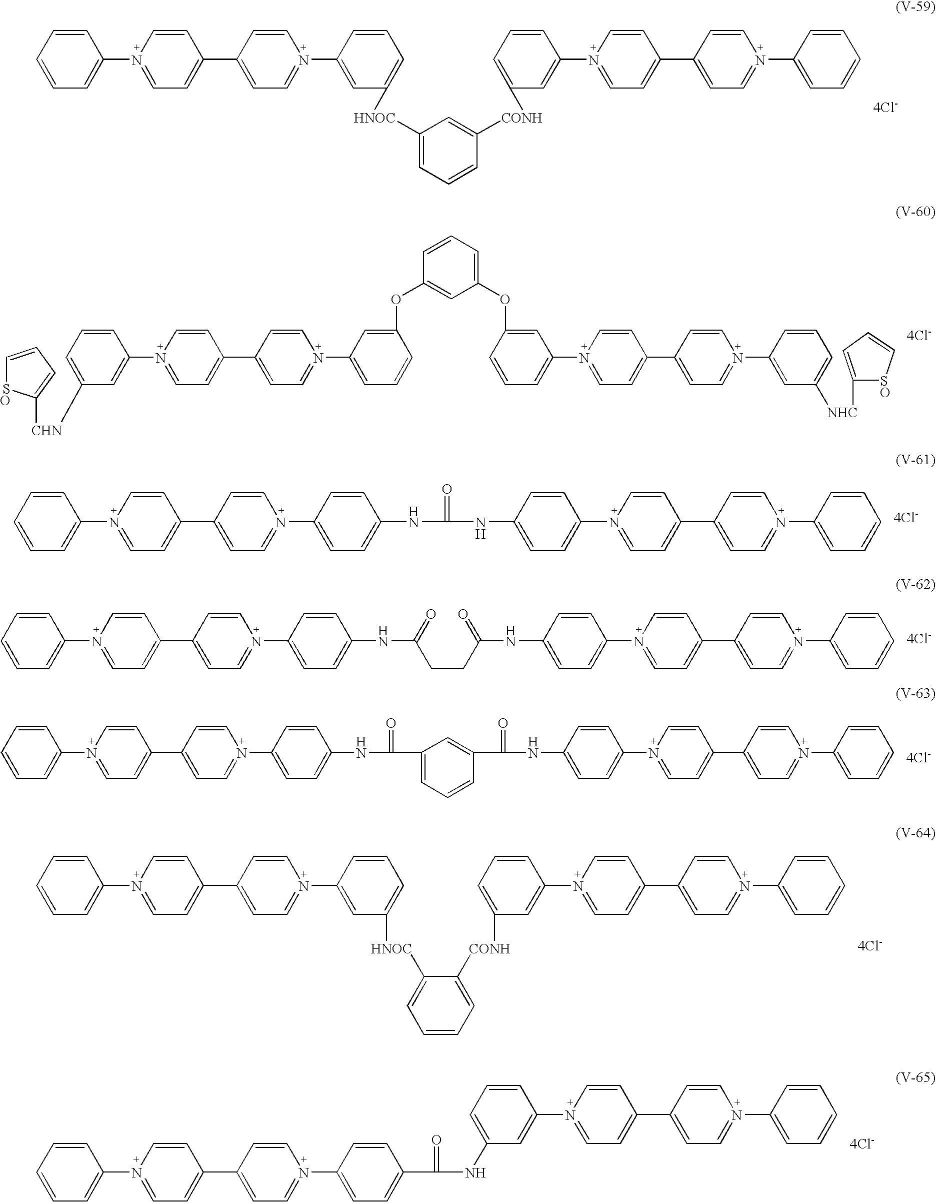

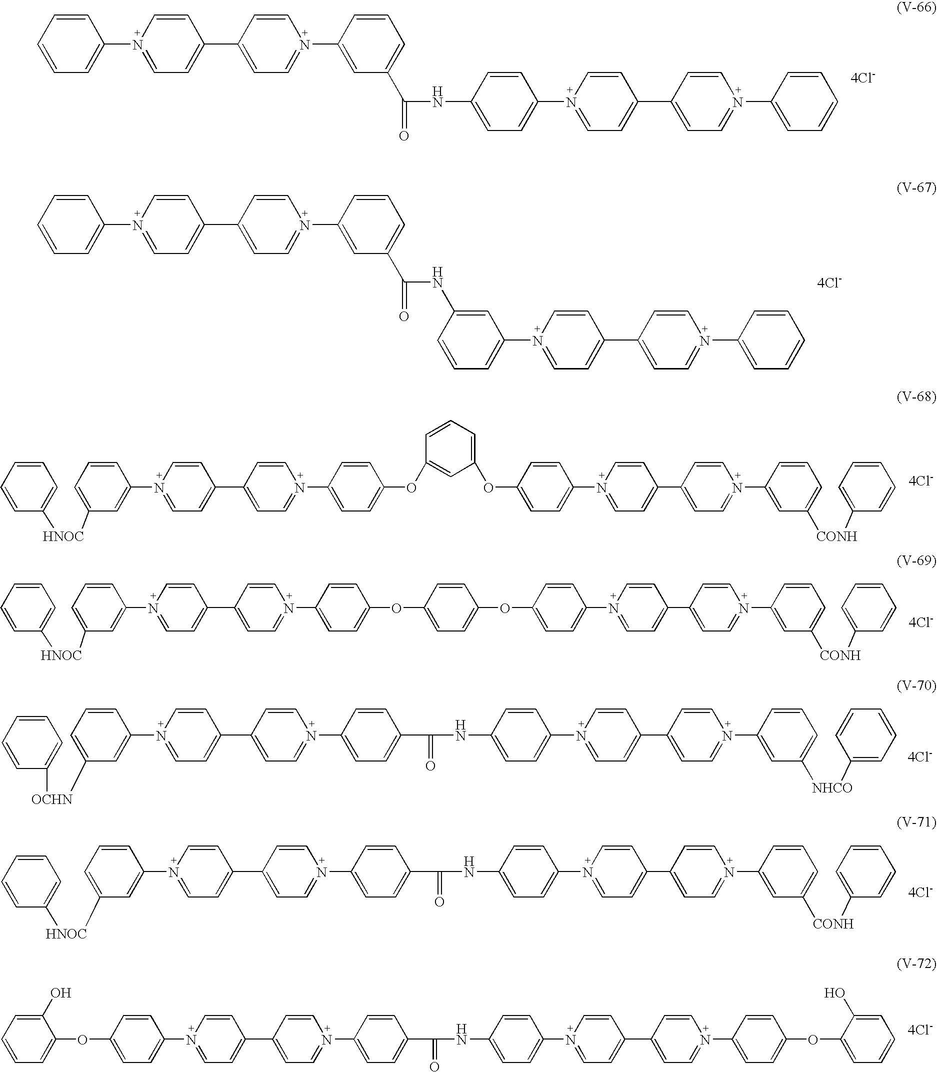

The present invention further relates to a method of manufacturing a bipyridinium compound denoted by general formula (7) by above-described steps (a) to (c). The details are as set forth above. Following step (c), the target compound can be separated by conducting the above-described known purification steps. Obtaining of the target compound can be confirmed by a known method such as NMR, mass spectrometry, or elemental analysis.

### Bipyridinium Compound

The present invention further relates to the asymmetric 4,4′-bipyridinium compound denoted by general formula (C). The details of general formula (C) are as set forth above. The bipyridinium compound denoted by general formula (C) can be obtained by above-described manufacturing method A. However, the bipyridinium compound denoted by general formula (C) of the present invention is not limited to compounds obtained by manufacturing method A.

The bipyridinium compound that has been obtained by the method of manufacturing a bipyridinium compound of the present invention and the bipyridinium compound denoted by general formula (C) of the present invention are useful as physiologically active drugs such as herbicides, electrochromic display materials, functional dyes such as optical recording dyes, and their constituent materials. Preferably, as described in Japanese Unexamined Patent Publication (KOKAI) No. 2004-188968 or English language family member US 2004/0166441 A1, which are expressly incorporated herein by reference in their entirety, they are employed as the paired cations of oxonol dyes serving as the antifading agents of optical disk dyes. Of these, the dye compound denoted by general formula (F) in which the bipyridinium compound denoted by general formula (C) serves as the paired cations affords good recording performance, storage properties and the like. The dye compound denoted by general formula (F) is described further below.

### Method of Manufacturing Dye Compound

The first aspect of the method of manufacturing a dye compound of the present invention is a method of manufacturing the dye compound denoted by general formula (D) by manufacturing the bipyridinium compound denoted by general formula (A) by manufacturing method A and reacting the bipyridinium compound that has been obtained with an anionic dye. The second aspect is a method of manufacturing the dye compound denoted by general formula (E) by manufacturing the bipyridinium compound denoted by general formula (B) by manufacturing method B and reacting the bipyridinium compound that has been obtained with an anionic dye.

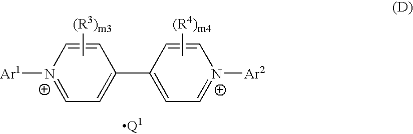

In general formula (D), Q¹ denotes a divalent anionic dye moiety, and Ar¹, Ar², R³, R⁴, m3 and m4 are respectively defined as in general formula (A). The details of Ar¹, Ar², R³, R¹, m3, and m4 are as set forth above.

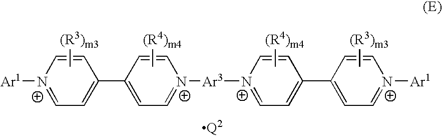

In general formula (E), Q² denotes two divalent anionic moieties, and Ar¹, Ar³, R³, R⁴, m3 and m4 are respectively defined as in general formula (B). The details of Ar¹, Ar³, R³, R⁴, me, and m4 are as set forth above.

The divalent anionic dye moiety is preferably an oxonol dye. The oxonol dye denoted by general formula (10) below is an example of an oxonol dye that is preferable as the divalent anionic dye moiety.

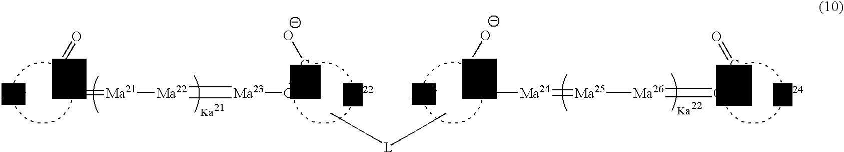

In general formula (10), Za²¹, Za²², Za²³ and Za²⁴ each independently denote an atom group forming an acidic nucleus. Examples thereof are described in: James ed., The Theory of the Photographic Process, 4th Ed., Macmillan Co., 1977, p. 198, which is expressly incorporated herein by reference in its entirety. Specific examples of nuclei, each of which is optionally substituted, are: pyrazole-5-one, pyrazolidine-3,5-dione, imidazoline-5-one, hydantoin, 2 or 4-thiohydantoin, 2-iminooxazolidine-4-one, 2-oxazoline-5-one, 2-thiooxazoline-2,4-dione, isorhodanine, rhodanine, thiophen-3-one, thiophen-3-one-1,1-dioxide, 3,3-dioxo[1,3]oxathiolan-5-one, indolin-2-one, indolin-3-one, 2-oxoindazolium, 5,7-dioxo-6,7-dihydrothiazolo[3,2-a]pyrimidine, 3,4-dihydroisoquinoline-4-one, 1,3-dioxane-4,6-dione (such as Meldrum's acid), barbituric acid, 2-thiobarbituric acid, cumarin-2,4-dione, indazoline-2-one, pyrido[1,2-a]pyrimidine-1,3-dione, pyrazolo[15-b]quinazolone, pyrazolopyridone, and five or six-membered carbon rings (such as hexane-1,3-dione, pentane-1,3-dione, indane-1,3-dione). Preferable nuclei are: pyrazole-5-one, pyrazolidine-3,5-dione, barbituric acid, 2-thiobarbituric acid, 1,3-dioxane-4,6-dione, 3,3-dioxo[1,3]oxathiolan-5-one, indanedione, pyrazolone, pyrazolinedione, and benzothiophenonedioxide. Each of Za²¹, Za²², Za²³, and Za²⁴ most preferably denotes optionally substituted 1,3-dioxane-4,6-dione.

In general formula (10), Ma²¹, Ma²², Ma²³, Ma²⁴, Ma²⁵ and Ma²⁶ each independently denote a substituted or unsubstituted methine group. Preferable substituents for substitution onto the methine group are: alkyl groups having 1 to 20 carbon atoms (such as methyl groups, ethyl groups, and isopropyl groups); halogen atoms (such as chlorine, bromine, iodine, and fluorine); alkoxy groups having 1 to 20 carbon atoms (such as methoxy groups, ethoxy groups, and isopropoxy groups), aryl groups having 6 to 26 carbon atoms (such as phenyl groups and 2-naphthyl groups), heterocyclic groups having 0 to 20 carbon atoms (such as 2-pyridyl groups and 3-pyridyl groups), aryloxy groups having 6 to 20 carbon atoms (such as phenoxy groups, 1-naphthoxy groups, and 2-naphthoxy groups); acylamino groups having 1 to 20 carbon atoms (such as acetylamino groups and benzoylamino groups); carbamoyl groups having 1 to 20 carbon atoms (such as N,N-dimethylcarbamoyl groups); sulfo groups; hydroxy groups; carboxy groups; alkylthio groups having 1 to 20 carbon atoms (such as methylthio groups); and cyano groups. They may also bond with other methine groups to form ring structures, or may bond with the atom groups denoted by Za²¹ to Za²⁴ to form ring structures.

Each of M²¹, Ma²², Ma²³, Ma²⁴, Ma²⁵, and Ma²⁶ preferably independently denotes an unsubstituted methine group, or methine group substituted with an ethyl, methyl, or phenyl group. An unsubstituted methine is further preferred.

Ka²¹ and Ka²² each independently denote an integer ranging from 0 to 3, with 2 being preferable. When Ka²¹ denotes 2 or 3, plural Ma²¹ and Ma²² present may be identical or different from each other, and when Ka²² denotes 2 or 3, Ma²⁵ and Ma²⁶ present may be identical or different from each other.

L denotes a divalent linking group that does not form a π-conjugated system with two bonds. The divalent linking group is not specifically limited, other than that it not form π-conjugated systems between the bonded chromophores. It preferably denotes a linking group having 0 to 100, more preferably 1 to 20, carbon atoms that is comprised of one, or a combination of two or more, selected from among alkylene groups (having 1 to 20 carbon atoms, such as methylene groups, ethylene groups, propylene groups, butylene groups, and pentylene groups); arylene groups (having 6 to 26 carbon atoms, such as phenylene groups and naphthylene groups); alkenylene groups (having 2 to 20 carbon atoms, such as ethenylene groups and propenylene groups), alkynylene groups (having 2 to 20 carbon atoms, such as ethynylene groups and propynylene groups); —CO—N(R¹⁰¹)—; —CO—O—; —SO₂—N(R¹⁰²)—; —SO₂—O—; —N(R¹⁰³)—CO—N(R¹⁰⁴)—; —SO₂—; —SO—; —S—; —O—; —CO—; —N(R¹⁰⁵)—; heterylene groups (having 1 to 26 carbon atoms, such as 6-chloro-1,3,5-triazyl-2,4-diyl groups and pyrimidine-2,4-diyl groups). R¹⁰¹, R¹⁰², R¹⁰³, R¹⁰⁴, and R¹⁰⁵ each independently denote a hydrogen atom, substituted or unsubstituted alkyl group, or substituted or unsubstituted aryl group. One or more of the linking groups denoted by L may be present between the two chromophores being linked, and multiple (preferably two) linking groups may bond together to form a ring.

L is preferably comprised of two alkylene groups (preferably ethylene groups) that are bonded together to form a ring. The case where a five or six-membered ring (preferably a cyclohexyl) is formed is preferred.

Preferable specific examples of the above oxonol dye are given below. However, the present invention is not limited thereto.

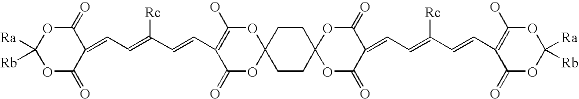

| Compound No. | Ra | Rb | Rc |
|---|---|---|---|
| C-1 | CH₃ | C₂H₅ | H |
| C-2 | CH₃ | C₄H₉-t | H |
| C-3 | C₂H₅ | C₃H₇-i | H |
| C-4 | C₂H₅ | C₂H₅ | H |
| C-5 | CH₃ | C₃H₇-n | H |
| C-6 | CH₃ | C₃H₇-n | CH₃ |
| C-7 | CH₃ | CH₂OCH₃ | H |
| C-8 | CH₃ | C₂H₄CO₂CH₃ | H |
| C-9 | CH₃ | C₂H₄CO₂C₂H₅ | H |
| C-10 | CH₃ | CH₃ | H |

C-11

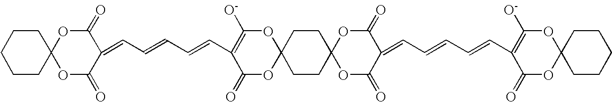

C-12

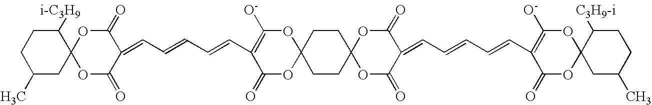

C-13

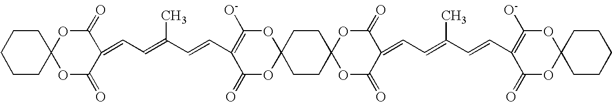

C-14

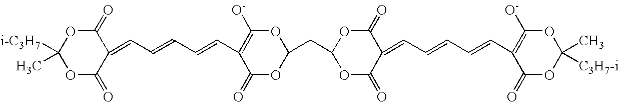

C-15

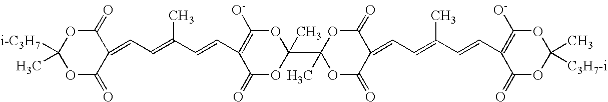

C-16

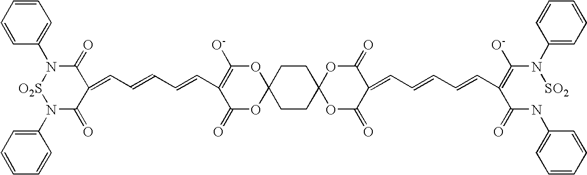

C-17

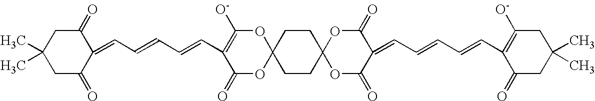

C-18

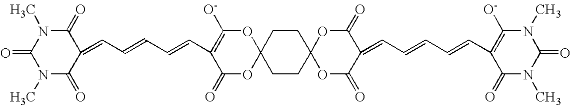

C-19

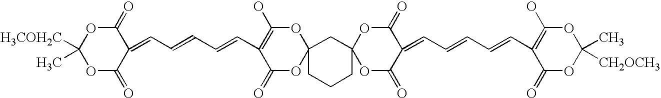

C-20

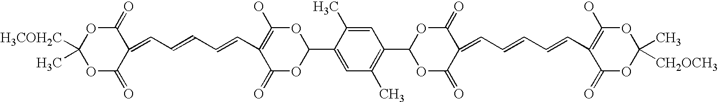

Common oxonol dyes can be synthesized by the condensation reaction of an appropriate active methylene compound and a methine source (a compound that is used to incorporate a methine group into a methine dye). For the details of such compounds, see Japanese Examined Patent Publication (KOKOKU) Showa Nos. 39-22069, 43-3504, 52-38056, 54-38129, 55-10059, and 58-35544; Japanese Unexamined Patent Publication (KOKAI) Showa Nos. 49-99620, 52-92716, 59-16834, 63-316853, and 64-40827; British Patent No. 1,133,986; and U.S. Pat. Nos. 3,247,127, 4,042,397, 4,181,225, 5,213,956, and 5,260,179.

European Patent EP 1,424,691 A2 discloses a method of synthesizing bis-type oxonol dyes.

In addition to the above-described oxonol dyes, dissociative azo dyes and azomethine dyes that have a chromophore group in the form of a dissociative group (hydroxyl group, amino group, or the like), and azo dyes, azomethine dyes, methine dyes, quinone dyes, diaryl or triarylmethane dyes, phthalocyanine dyes, indigo dyes, condensed ring dyes, styryl dyes, spiropyrans, spirooxazine derivatives, diarylethene derivatives, squalium, croconium derivatives that have a dissociative group and a chromophore group as substituents can be employed as the anionic dye moieties (Q¹ and Q²) in general formulas (D) and (E). Examples of specific structures are the dyes described in Okawara M., Matsuoka M., Hirajima T., Kitao T. (Kodansha), “Functional Dyes.”, which is expressly incorporated herein by reference in its entirety.

In the first aspect of the method of manufacturing a dye compound of the present invention, a bipyridinium compound is synthesized by manufacturing method A, and the bipyridinium compound obtained is optionally purified or the like and then reacted with an anionic dye to synthesize the dye compound denoted by general formula (D). In the second aspect of the method of manufacturing a dye compound of the present invention, a bipyridinium compound is synthesized by manufacturing method B, and the bipyridinium compound obtained is optionally purified or the like and then reacted with an anionic dye to synthesize the dye compound denoted by general formula (E). The reaction of the bipyridinium compound with the anionic dye can be readily conducted by anion-exchanging a salt (hydrochloride or the like) of the bipyridinium compound. As an example of the method of exchanging anion dyes, a salt of the bipyridinium compound is dissolved in a polar solvent such as water or an alcohol, an anionic dye solution is added, and the mixture is stirred with heating (40 to 100° C., for example) and reacted for 0.5 to 2 hours. Following the reaction, the target compound can be precipitated out as crystals. The dye compound obtained can be purified by known methods. A known analysis method such as NMR can be used to confirm that the targeted dye compound has been obtained.

Examples of combinations of bipyridinium compounds and oxonol dyes in dye compounds obtained by the method of manufacturing a dye compound of the present invention are given below. However, the present invention is not limited thereto. The cation moiety in Table 2 refers to the cation moiety contained in the compound of the example compound.

TABLE 1

| Compound No. | Anion moiety | Cation moiety |
|---|---|---|
| D-1 | C-5 | V-1 |
| D-2 | C-9 | V-2 |
| D-3 | C-4 | V-3 |
| D-4 | C-9 | V-4 |
| D-5 | C-9 | V-5 |
| D-6 | C-11 | V-6 |
| D-7 | C-5 | V-7 |
| D-8 | C-5 | V-8 |
| D-9 | C-9 | V-9 |
| D-10 | C-5 | V-10 |
| D-11 | C-5 | V-11 |
| D-12 | C-9 | V-12 |
| D-13 | C-5 | V-13 |
| D-14 | C-9 | V-14 |
| D-15 | C-9 | V-15 |
| D-16 | C-11 | V-16 |
| D-17 | C-9 | V-17 |
| D-18 | C-5 | V-18 |
| D-19 | C-9 | V-19 |
| D-20 | C-5 | V-20 |
| D-21 | C-5 | V-21 |
| D-22 | C-5 | V-22 |
| D-23 | C-9 | V-23 |
| D-24 | C-5 | V-24 |

TABLE 2

| Compound No. | Anion moiety | Cation moiety |
|---|---|---|
| D-25 | C-5 | V-31 |
| D-26 | C-9 | V-32 |
| D-27 | C-4 | V-33 |
| D-28 | C-9 | V-34 |
| D-29 | C-9 | V-35 |
| D-30 | C-11 | V-36 |
| D-31 | C-9 | V-37 |
| D-32 | C-9 | V-38 |
| D-33 | C-9 | V-39 |
| D-34 | C-5 | V-40 |
| D-35 | C-5 | V-41 |
| D-36 | C-9 | V-42 |
| D-37 | C-5 | V-43 |
| D-38 | C-9 | V-44 |
| D-39 | C-9 | V-45 |
| D-40 | C-11 | V-46 |
| D-41 | C-9 | V-47 |
| D-42 | C-5 | V-48 |
| D-43 | C-9 | V-49 |
| D-44 | C-5 | V-50 |
| D-45 | C-5 | V-51 |
| D-46 | C-5 | V-52 |
| D-47 | C-9 | V-53 |
| D-48 | C-5 | V-54 |
| D-49 | C-5 | V-55 |
| D-50 | C-5 | V-56 |
| D-51 | C-5 | V-57 |
| D-52 | C-4 | V-58 |
| D-53 | C-5 | V-59 |
| D-54 | C-9 | V-60 |
| D-55 | C-5 | V-61 |
| D-56 | C-9 | V-62 |
| D-57 | C-5 | V-63 |
| D-58 | C-9 | V-64 |
| D-59 | C-5 | V-65 |
| D-60 | C-5 | V-66 |
| D-61 | C-5 | V-67 |
| D-62 | C-5 | V-68 |
| D-63 | C-5 | V-69 |
| D-64 | C-9 | V-70 |
| D-65 | C-5 | V-71 |
| D-66 | C-9 | V-72 |

### Dye Compound

The present invention further relates to the dye compound denoted by general formula (F) below.

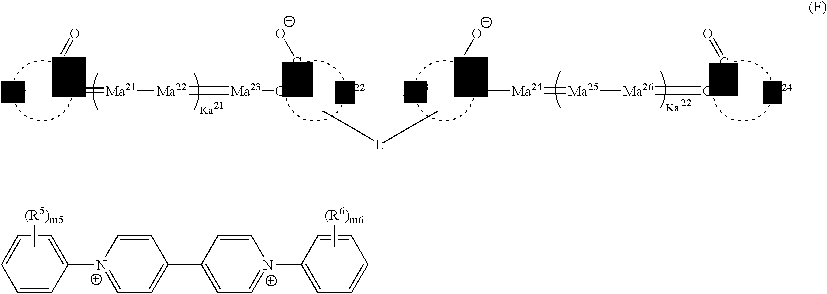

In general formula (F), Za²¹, Za²², Za²³, Za²⁴, Ma²¹, Ma²², Ma²³, Ma²⁴, Ma²⁵, Ma²⁶, L, Ka²¹ and Ka²² are respectively defined as in general formula (10). The details thereof are as set forth above. R⁵, R⁶, m5 and m6 are respectively defined as in general formula (C). The details thereof are as set forth above.

The dye compound denoted by general formula (F) can be readily obtained by anion-exchanging a salt (such as a hydrochloride) of the bipyridinium compound denoted by general formula (C) above. The compounds given in Table 1 are specific examples of the dye compound denoted by general formula (F).

### EXAMPLES

The present invention will be described in detail below based on examples. However, the present invention is not limited to the examples.

### Example 1

### Synthesis of Dye Compound D-7

Example compound D-7 was synthesized by the following scheme.

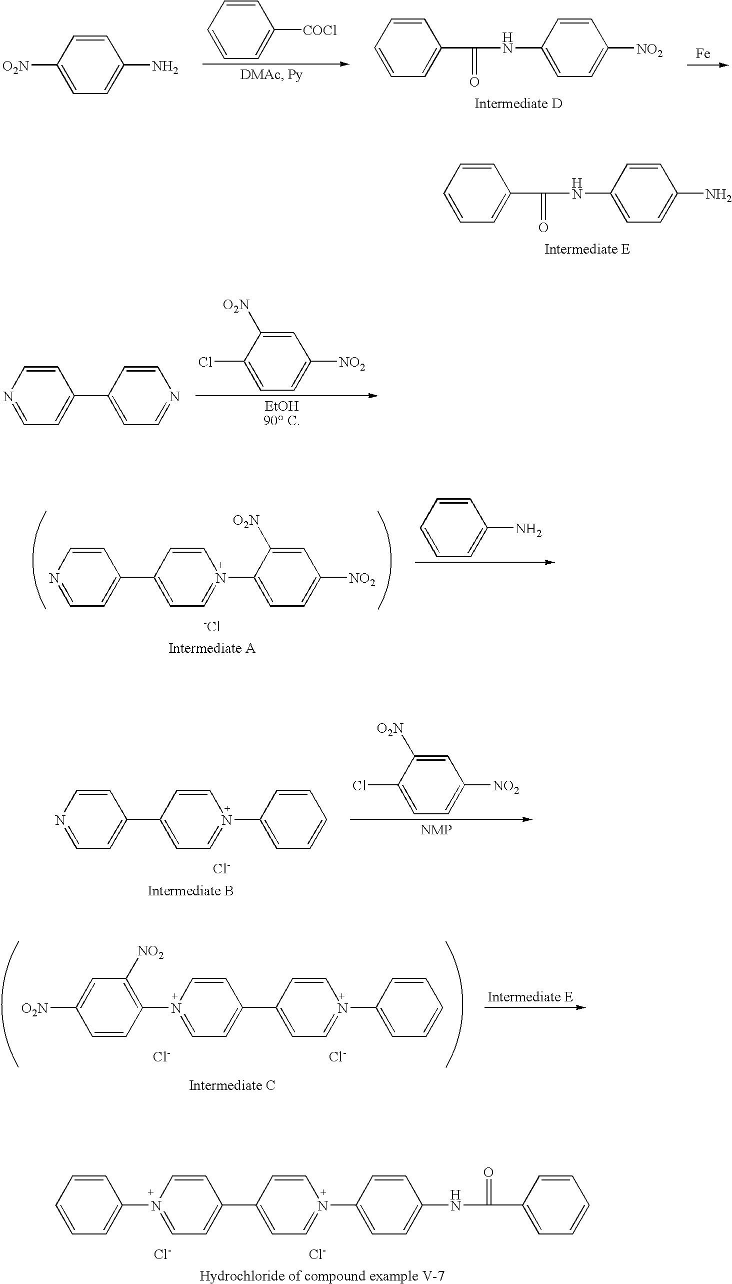

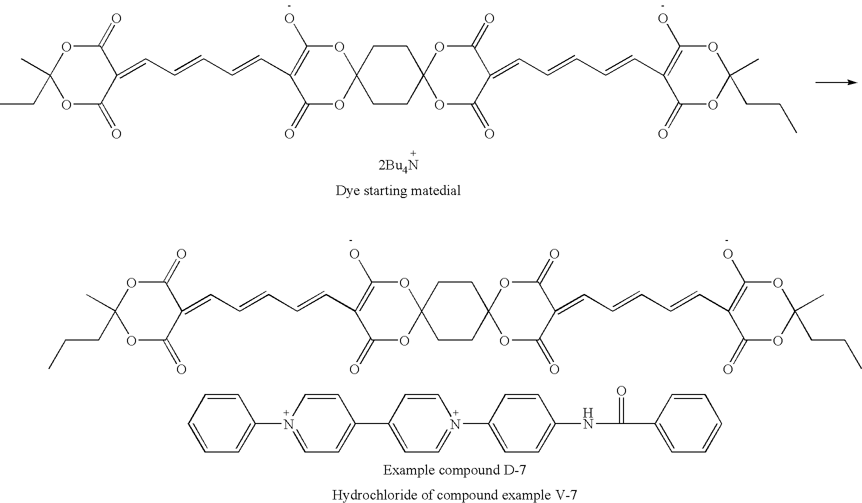

(1) Synthesis of Hydrochloride of Compound Example V-7

(i) Synthesis of Intermediate D

A 3.0 g quantity of 4-nitroaniline was dissolved in 20 mL of dimethylformamide. To this solution was added 1.82 g of pyridine and the mixture was stirred at room temperature (25° C.). Next, 2.95 g of benzoyl chloride was gradually added and the mixture was stirred for 4 hours. Following completion of the reaction, the mixture was poured into 400 mL of water. The precipitating crystals were collected by filtration, washed with a 1 percent hydrochloride aqueous solution, washed with acetonitrile, and dried, yielding 4.52 g of intermediate D.

(ii) Synthesis of Intermediate E

To 60 mL of isopropanol and 8 mL of water were added 0.52 g of ammonium chloride and 7.3 g of reduced iron, and the mixture was refluxed with heating for 30 minutes. Next, while conducting hot refluxing, 4.0 g of intermediate D was gradually added. After continuing hot refluxing for another one hour, the insoluble material was removed by filtration while hot. The isopropanol solution obtained was poured into 500 mL of water. The precipitating crystals were recovered by filtration and dried, yielding 1.2 g of intermediate E.

(iii) Synthesis of Intermediate B

To 15 g of 4,4′-bipyridyl and 13 g of 1-chloro-2,4-dinitrobenzene was added 100 mL of acetonitrile and the mixture was refluxed with heating (reaction temperature 85° C.) for 3 hours. Next, 5 g of aniline was added dropwise while conducting refluxing with heating. Refluxing with heating (reaction temperature 85° C.) was continued for another 2 hours. When the reaction had ended, the mixture was cooled. Water and ethyl acetate were added, and the mixture was extracted with water. A 200 mL quantity of acetone was added dropwise to the extraction. The precipitating crystals were recovered by filtration and dried, yielding 18 g of intermediate B.

¹H-NMR data of intermediate B (d⁶-DMSO): 9.59(d, 2H), 9.04(d,2H), 8.87(d,2H), 8.40(d,2H), 7.97-7.99(m,2H), 7.77-7.80(m,3H)

(iv) Synthesis of Hydrochloride of Compound Example V-7

To 3 g of intermediate B and 7 g of 1-chloro-2,4-dinitrobenzene was added 5 mL of butanol and the mixture was stirred with heating for 3 hours in an oil bath with an external temperature of 85° C. After cooling, water, ethanol, and toluene were added and the mixture was extracted with water. Toluene was added to the extraction, the mixture was extracted again with water, 1.4 g of intermediate E was added, and the mixture was stirred with heating for 9 hours at 90° C. When the reaction had ended, the mixture was diluted with 30 mL of acetonitrile. The reaction solution was then poured into 400 mL of ethyl acetate. The precipitating crystals were recovered by filtration and dried, yielding 3.4 g of the hydrochloride of compound example V-7.

¹H-NMR data of hydrochloride of compound example V-7 (d⁶-DMSO): 10.90(s, 1H), 9.76-9.69(m,4H), 9.16-9.08(m,4H), 8.28-7.79(m,10H), 7.70-7.53(m,4H)

(2) Synthesis of Dye Compound D-7 (Formation of Salt)

A 0.45 g quantity of the hydrochloride of V-7 obtained above was dissolved with heating in 6.5 mL of methanol. To this solution was added 1.24 g of dye starting material and the mixture was stirred for 30 min at 50° C. The mixture was cooled and then stirred for 1 hour at room temperature. The precipitating crystals were recovered by filtration and washed with methanol. A 13 mL quantity of methanol was then added. The mixture was stirred for 30 min at 50° C., cooled, and then stirred for 1 hour at room temperature. The crystals obtained were recovered by filtration, washed with methanol, and dried, yielding 0.9 g of compound D-7.

¹H-NMR data of compound example D-7(d⁶-DMSO): 10.53(s,1H), 9.75(s(br),4H), 9.11(s(br),4H), 8.37(s(br),2H), 8.15(s(br),2H), 7.98(s(br),2H), 7.83-7.39(m,14H), 7.39-7.11(m,4H), 1.99(s,8H), 1.82-1.74(m,4H), 1.52(s,6H), 1.46-1.34(m,4H), 0.89(t,6H)

### Example 2

### Synthesis of Dye Compound D-8

Synthesis of Dye Compound (D-8)

Example compound D-8 was synthesized by the following scheme.

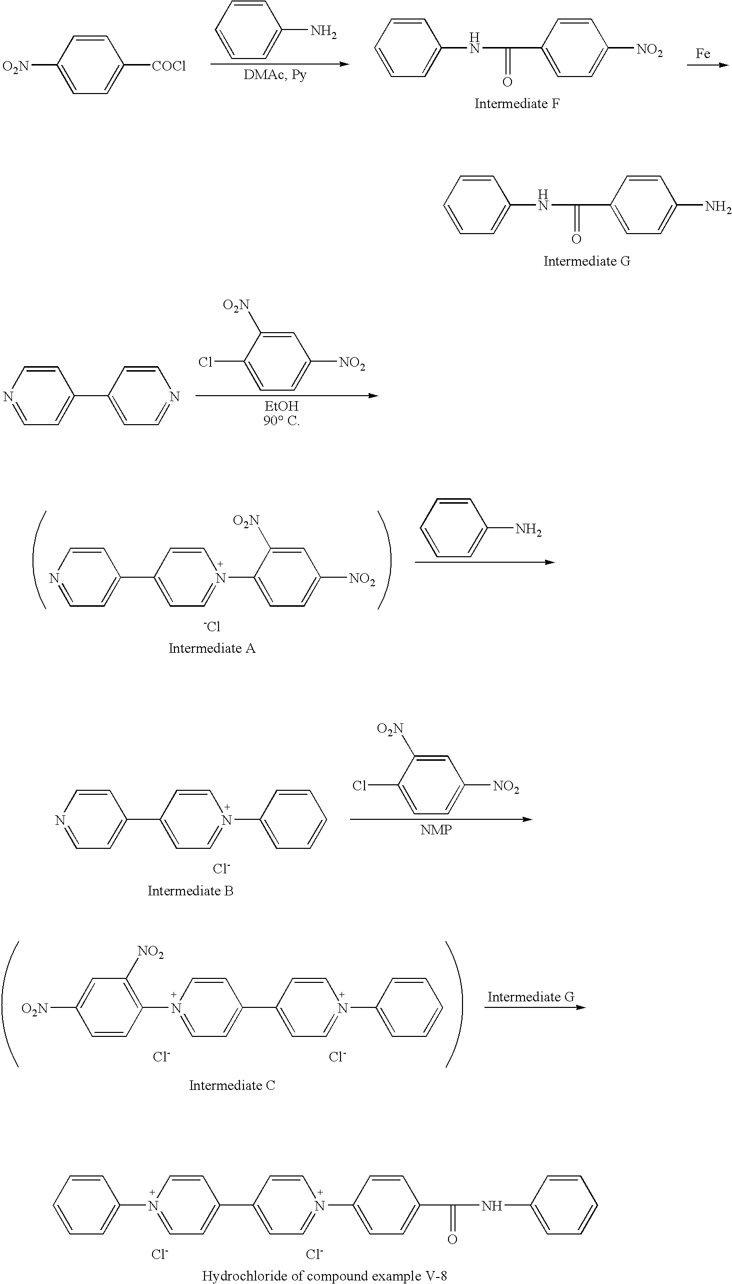

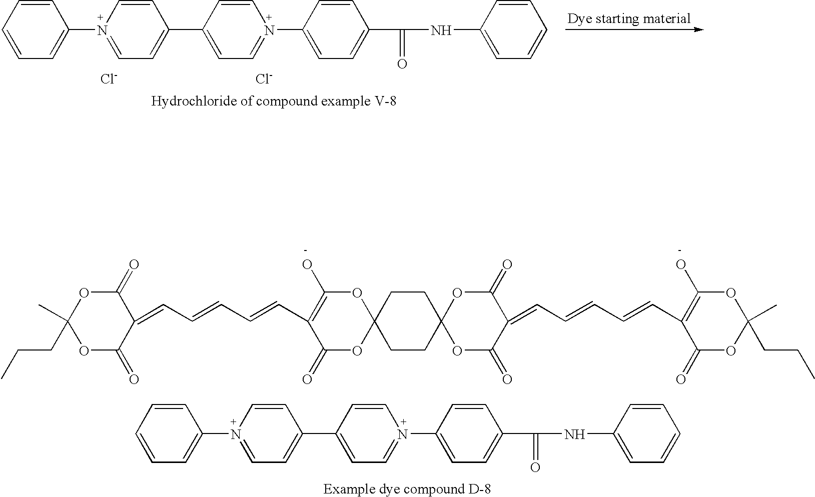

(1) Synthesis of Intermediates G and F

The starting materials in the synthesis of intermediates D and E in Example 1 were changed to synthesize intermediates F and G.

(2) Synthesis of Hydrochloride of Compound Example V-8

A 1.02 g quantity of the hydrochloride of compound example V-8 was synthesized by employing intermediates F and G in place of intermediates D and E by a method identical to that set forth above.

¹H-NMR data of hydrochloride of compound example V-8 (d⁶-DMSO): 9.68-9.57(m,4H), 9.00-8.91(m,4H), 8.36(d,2H), 8.10(d,2H), 7.98-7.94(m,2H), 7.86-7.78(m,3H), 7.78-7.72(m,2H), 7.41(t,2H), 7.20(t,1H)

(3) Synthesis of Dye Compound D-8 (Formation of Salt)

Chlorine anions of the hydrochloride obtained were anion exchanged in the manner described in the above-described example, yielding 0.9 g of compound D-8 comprising oxonol dye as paired anions.

¹H-NMR data of hydrochloride of compound example D-8 (d⁶-DMSO): 10.72(s,1H), 9.70(s(br),4H), 9.08(s(br),4H), 8.21-8.16(m,2H), 8.05-7.95(m,6H), 7.87-7.70(m,3H), 7.71-7.48(m,8H), 2.00(s,8H), 1.83-1.75(m,4H), 1.53(s,6H), 1.46-1.33(m,4H), 0.90(t,6H)

### Example 3

An example of synthesis of the above-described hydrochloride of compound example V-7 by a sequential method in which the intermediates are separated will be described next.

(1) Synthesis of Hydrochloride of Compound Example V-7

(i) Synthesis of Intermediate A

A 15 g quantity of 4,4′-bipyridyl was dissolved in 100 mL of acetone. To this solution was added 13.2 g of 1-chloro-2,4-dinitrobenzene. The mixture was stirred for 15 minutes at room temperature, and then refluxed with heating for another 15 minutes. When the reaction had ended, the mixture was cooled to room temperature, and the precipitating crystals were recovered by filtration under reduced pressure. The crystals that were fmally obtained were washed with acetone and dried, yielding 18.8 g of intermediate A.

¹H-NMR data of intermediate A (d⁶-DMSO): 9.62(d,2H), 9.17(s,1H), 8.94-903(m, 5H), 8.49(d, 1H), 8.21(d,2H)

(ii) Synthesis of Intermediate B

A 14.4 g quantity of intermediate A was suspended in 100 mL of acetonitrile, 4.6 g of aniline was added, and the mixture was refluxed with heating for 7 hours. When the reaction had ended, the mixture was cooled to room temperature. The precipitating crystals were recovered by filtration, washed with acetonitrile, and dried. A 20 mL quantity of methanol was added to the crude crystals obtained, which were completely dissolved with heating. To this solution was added 200 mL of ethyl acetate and the mixture was stirred for one hour at room temperature. The crystals obtained were recovered by filtration, yielding 10.4 g of intermediate B.

(iii) Synthesis of Intermediate C

To 3 g of intermediate B and 7 g of 1-chloro-2,4-dinitrobenzene was added 5 mL of N-methylpyrrolidone and the mixture was heated for 9 hours in an oil bath with an external temperature of 110° C. When the reaction had ended, the mixture was cooled to room temperature. The precipitating crystals were recovered by filtration, washed with N-methylpyrrolidone, washed again with ethyl acetate, and dried, yielding 3.7 g of intermediate C.

¹H-NMR data of intermediate C (d⁴-MeOD): 9.65(d,2H), 9.59(d,2H), 9.32(s, 1H), 9.08(d, 2H), 8.96-9.00(m,3H), 8.44(d,1H), 7.95-7.99(m,2H), 7.81-7.84(m, 3H)

(vi) Synthesis of Hydrochloride of Compound Example V-7

A 1.6 g quantity of intermediate C was dissolved in 7.5 mL of dimethylformamide, 0.6 g of intermediate E was added, and the mixture was stirred with heating for 5 hours at 90° C. When the reaction had ended, the reaction solution was cooled to room temperature and diluted with 30 mL of acetonitrile. The reaction solution was then poured into 400 mL of ethyl acetate. The precipitating crystals were recovered by filtration and dried, yielding 1.45 g of hydrochloride of compound example V-7.

Examples 1 to 3 permitted the synthesis of asymmetric aryl-substituted bipyridinium compounds under mild reaction conditions without placing a heavy burden on the environment or on operations and while employing a minimum of harmful solvents, reactants, and the like. Example 1 made it possible to obtain the targeted compound rapidly and without separation of intermediates A and C. Example 2 made it possible to obtain the targeted compound rapidly while similarly omitting the step of separating the intermediates. Example 3 made it possible to obtain the targeted compound in integrated fashion, without separating the intermediates, and with a shorter reaction time and a higher yield than in Example 1. It will be understood that the manufacturing process was thus simplified.

### Example 4

### Synthesis of Bis-Type Bipyridinium Compound V-31

The hydrochloride of example compound V-31 was synthesized by the following scheme.

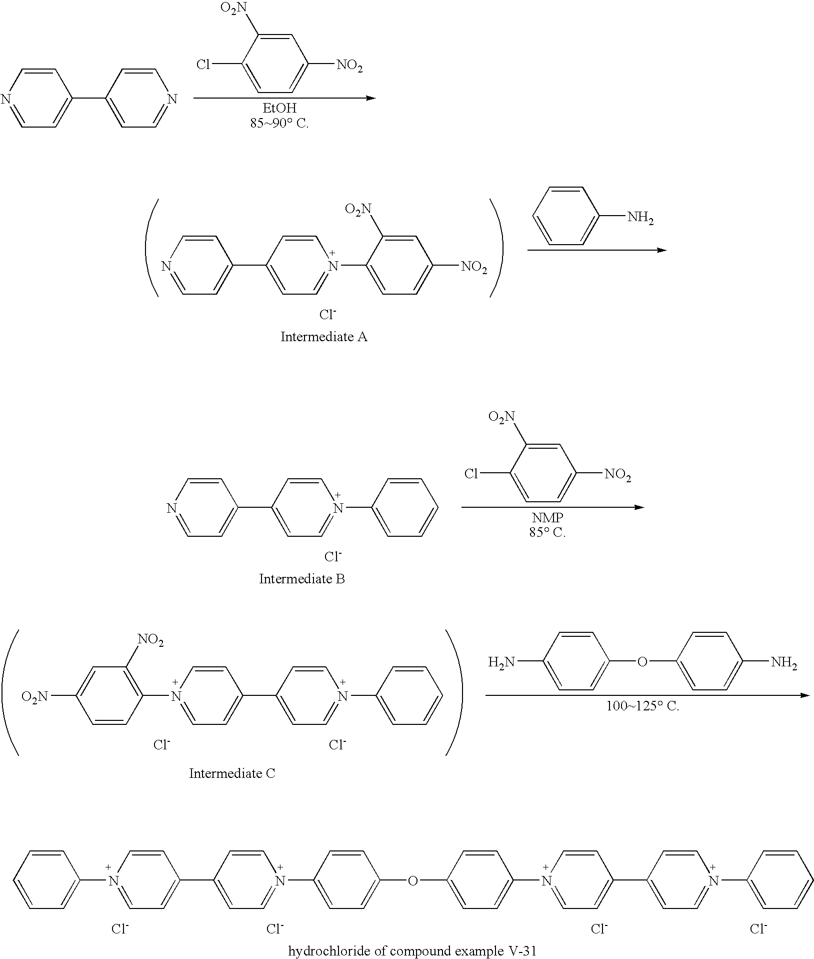

(i) Synthesis of Intermediate B

To 52 g of 4,4′-bipyridyl and 45 g of 1-chloro-2,4-dinitrobenzene was added 33 mL of acetonitrile and the mixture was refluxed with heating (reaction temperature 85° C.) for 3 hours. Next, 25 g of aniline was added dropwise while refluxing with heating. The mixture was further refluxed with heating (reaction temperature 85° C.) for 2 hours. When the reaction had ended, the mixture was cooled, water and ethyl acetate were added, and the mixture was extracted with water. To the extraction was added dropwise 495 mL of acetone, and the precipitating crystals were recovered by filtration and dried, yielding 42 g of intermediate B.

(ii) Synthesis of Hydrochloride of Compound Example V-31

To 25 g of intermediate B and 57 g of 1-chloro-2,4-dinitrobenzene was added 8 mL of N-methylpyrrolidone and the mixture was stirred with heating for 3 hours in an oil bath with an external temperature of 75° C. The mixture was cooled. Water, ethanol, and toluene were added, and the mixture was extracted with water. Toluene was added to the extraction, the mixture was again extracted with water, 9 g of 4,4′-diaminodiphenylether was added, and the mixture was stirred for 6 hours with heating at 90° C. When the reaction had ended, it was poured into 240 mL of acetonitrile. The precipitating crystals were recovered by filtration and dried, yielding 41 g of hydrochloride of compound example V-31.

### Example 5

### Synthesis of Hydrochloride of Compound Example V-57

The hydrochloride of compound example V-57 was synthesized by the following scheme.

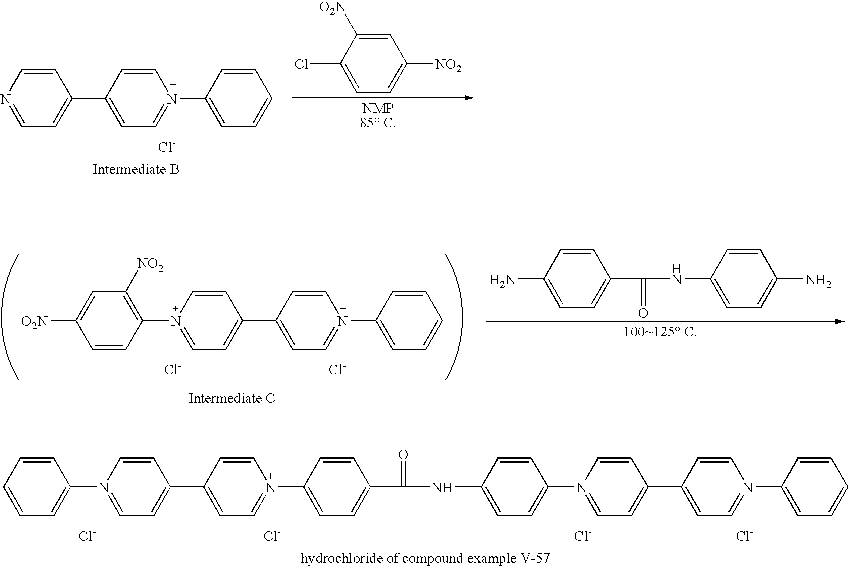

To 30 g of intermediate B synthesized by the same operation as in Example 1 and 68 g of 1-chloro-2,4-dinitrobenzene was added 10 mL of N-methylpyrrolidone and the mixture was stirred with heating for 3 hours in an oil bath with an external temperature of 85° C. The mixture was cooled. Water, ethanol, ethylene glycol, and toluene were added, and the mixture was extracted with water. Toluene was added to the extraction and the mixture was extracted again with water. To the extraction was added 13 g of 4,4′-diaminobenzanilide and the mixture was stirred with heating for 9 hours at 125° C. When the reaction had ended, the reaction solution was poured onto 290 mL of acetonitrile. The precipitating crystals were recovered by filtration and dried, yielding 27 g of hydrochloride of compound example V-57.

¹H-NMR data of compound example V-57 (CD₃OD): 9.68-9.57(m,8H), 9.00-8.92(m,8H), 8.42(d,2H), 8.29(d,2H), 8.17(d,2H), 8.02-7.93(m,6H), 7.83-7.80 (m,6H)

Evaluation Methods

The coating surface properties and solution stability over time of example compounds D-7 and D-8 and the comparative examples set forth below were evaluated by the following methods:

### (1) Evaluation of Suitability to Spin Coating

The dyes (0.3 g) listed in Table 3 were dissolved in 10 mL of 2,2,3,3-tetrafluoropropanol (TFP). The solutions were spin coated onto polycarbonate substrates and visually inspected for coating streaks. The results are given in Table 3.

### (2) Test of Dissolution Stability Over Time

The dyes listed in Table 3 were prepared as 5.0 weight percent solutions of 2,2,3,3-tetrafluoropropanol and left standing for one week at 20° C. Crystal precipitation was visually determined. The results are given in Table 3.

TABLE 3

| Dye compound | Coating streaks | Dissolution stability over time |
|---|---|---|
| D-7 | Not observed | ⊚ |
| D-8 | Not observed | ⊚ |
| Comparative compound A | Some coating streaks were observed. | Δ |
| Comparative compound B | Observed | ⊚ |
| Comparative compound C | Some coating streaks were observed. | X |

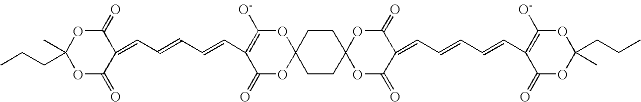

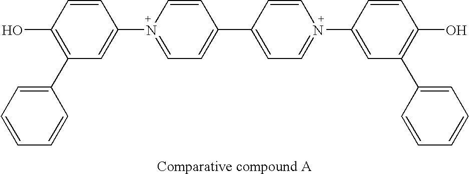

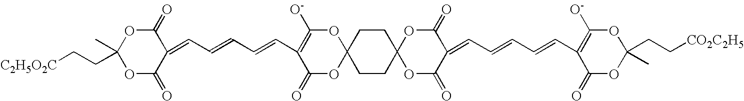

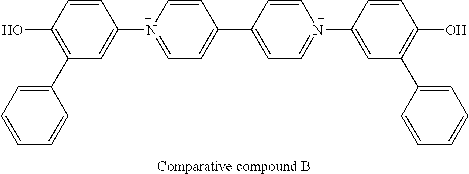

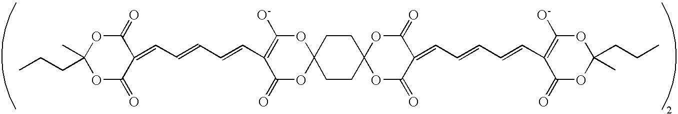

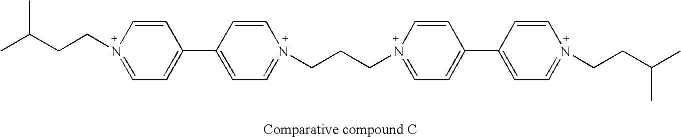

⊚: Absolutely no crystal precipitation was observed.
◯: No crystal precipitation was observed.
Δ: Slight crystal precipitation was observed.
X: Substantial crystal precipitation was observed.

As will be clear from the results in Table 3, example compounds D-7 and D-8 tended not to develop coating streaks and exhibited excellent dissolution stability over time. This trend was similarly observed using other dye compounds of the present invention.

Performance Evaluation of an Optical Information Recording Medium

Preparation of Optical Information Recording Medium

Polycarbonate resin was shaped by injection molding into supports 120 mm in diameter and 0.6 mm in thickness with spiral grooves (130 nm deep, 310 nm wide, with a track pitch of 0.74 micrometer). A coating liquid comprised of 1.25 g of comparative compound A dissolved in 100 mL of 2,2,3,3-tetrafluoropropanol was prepared. This coating liquid was applied by spin coating to the grooved surface of the support, forming a dye layer.

Next, silver was sputtered onto the dye-coated surface to form a reflective film about 150 nm in thickness. UV-curable resin (Daicure Clear SD640 made by Dainippon Ink and Chemicals, Inc.) was employed as bonding agent to bond the support to a dummy support 0.6 mm in thickness to prepare a DVD-R disk.

Evaluation of Optical Information Recording Medium

Using a DDU-1000 and a multisignal generator (made by Pulstech Corp., laser wavelength 660 nm, numerical aperture 0.60), an 8-16 modulated signal was recorded at a transfer rate speed multiple of 1 (11.08 Mbps), speed multiple of 8 (88.64 Mbps), and speed multiple of 10 (110.8 Mbps).

Table 4 shows the recording strategies employed. One type was employed for recording at speed multiples of 1 and 10, and two types with greatly differing pulse widths were employed for recording at a speed multiple of 8.

The recording power was set to the level that minimized jitter for each medium. Subsequently, reproduction was conducted with a laser having the same wavelength as the recording laser and sensitivity and jitter were measured. The results are given in Table 5. It proved possible to establish good recording strategies.

TABLE 2
Recording strategies

| Recording rate | 1X | 8X | 8X | 10X |
|---|---|---|---|---|
| Recording strategy | A | B | C | D |
| 3Ttop | 1.55 | 2.55 | 1.85 | 2.75 |
| 4Ttop | 1.50 | 2.92 | 2.12 | 3.20 |
| nTtop | 1.55 | 1.70 | 1.30 | 1.90 |
| Tmp | 0.85 | — | — | — |
| nTwt | — | 0.50 | −0.30 | 0.55 |
| nTip | — | 1.40 | 0.80 | 1.40 |
| 3-nTld | — | −0.03 | −0.05 | −0.03 |
| 3Tdoop | — | −0.15 | −0.05 | −0.15 |
| 4Tdoop | — | 0.20 | 0.35 | 0.20 |
| nTdop | — | 0.00 | 0.00 | 0.00 |
| 5Ttop2 | — | −0.15 | −0.05 | −0.20 |
| 5Tip2 | — | −0.10 | −0.15 | −0.20 |
| 5Tdp2 | — | 0.00 | 0.00 | 0.00 |
| P0/Pm | — | 1.48 | 1.58 | 1.36 |

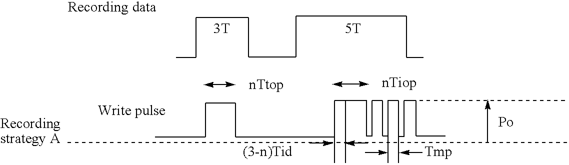

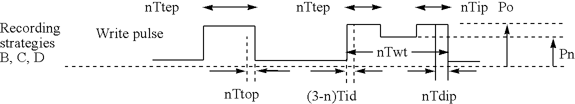

TABLE 5

Example

| | | | | |
|---|---|---|---|---|
| Recording rate | 1X | 8X | 8X | 10X |
| Recording | A | B | C | D |
| strategy | | | | |
| Optimal | 10 | 26 | 32 | 32 |
| recording | | | | |
| power (mW) | | | | |
| Reflectance | 51.2 | 52.1 | 51.8 | 51.7 |
| Jitter(%) | 6.7 | 6.8 | 6.9 | 6.9 |
| 14T | 0.52 | 0.71 | 0.77 | 0.78 |
| modulation | | | | |
| degree | | | | |
| PI error | 23 | 18 | 11 | 16 |
| AR (%) | 50 | 35 | 28 | 26 |

A 12× recording strategy was then set for comparative compound A in the same manner as in Tables 4 and 5. DVD-R disks were then prepared in the same manner as set forth above with the exception that the various dyes listed in Table 6 were employed instead of comparative compound A. A 12× recording and reproduction test was conducted using the various disks. The results are given in Table 6.

TABLE 6

| Dye compound | Sensitivity (mW) | Jitter (%) |
|---|---|---|
| D-7 | 35 | 6.5 |
| D-8 | 33 | 6.6 |
| Comparative | 38 | 7.2 |
| compound A | | |
| Comparative | 42 | 7.6 |
| compound B | | |
| Comparative | 51 | 8.6 |
| compound C | | |

As shown in Table 6, the DVD-R disks in which the dye compounds synthesized in Examples were employed as recording layer dyes exhibited excellent sensitivity and jitter.

The method of manufacturing a bipyridinium compound of the present invention affords a short reaction time, achieves good yields, and yields the target compound in an integrated manner without separation of intermediates, thereby simplifying the manufacturing process. Further, the method of manufacturing a bipyridinium compound of the present invention permits the synthesis of asymmetric 4,4′-bipyridinium compounds under mild reaction conditions, placing little burden on the environment or operations, by a clean method employing a minimum of harmiful solvents, reactants, and the like.

The superiority and usefulness of the manufacturing method of the present invention are thus clearly evident. Further, DVD-R dyes employing asymmetric viologen have good disk characteristics. In particular, they afford good solution stability over time and good coating surface properties that are important during disk manufacturing.

Although the present invention has been described in considerable detail with regard to certain versions thereof, other versions are possible, and alterations, permutations and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

What is claimed is:

1. A method of manufacturing a bipyridinium compound denoted by general formula (A) comprising steps of:
(a) manufacturing a bipyridinium compound denoted by general formula (3) by reacting a bipyridine compound denoted by general formula (1) with a (hetero)arylhalogen compound denoted by general formula (2) in a solvent;
(b) manufacturing a N-aryl-substituted bipyridinium compound denoted by general formula (5) by reacting the bipyridinium compound denoted by general formula (3) that has been obtained in step (a) with an amine compound denoted by general formula (4) in a solvent;
(c) manufacturing a bipyridinium compound denoted by general formula (7) by reacting the N-aryl-substituted bipyridinium compound denoted by general formula (5) that has been obtained in step (b) with a (hetero)arylhalogen compound denoted by general formula (6) in a solvent; and
(d) manufacturing the bipyridinium compound denoted by general formula (A) by reacting the bipyridinium compound denoted by general formula (7) that has been obtained in step (c) with an amine compound denoted by general formula (8) in a solvent. 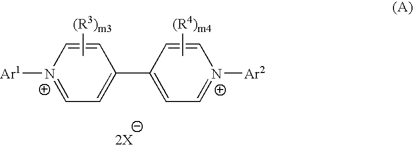
In general formula (A), Ar¹ and Ar² each independently denote an optionally substituted (hetero)aryl group, R³ and R⁴ each independently denote a substituent that may form a ring with a pyridine ring to which the substituent substitutes, m3 and m4 each independently denote an integer ranging from 0 to 4, X denotes a halogen atom or RSO₃, and R denotes an optionally substituted aryl group or alkyl group. When m3 denotes an integer ranging from 2 to 4, plural R³ present may be identical or different from each other and when m4 denotes an integer ranging from 2 to 4, plural R⁴ present may be identical or different from each other. 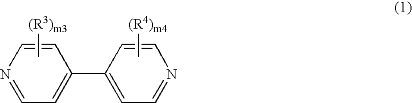
In general formula (1), R³, R⁴, m3 and m4 are respectively defined as in general formula (A).
R¹—X (2)
In general formula (2), R¹ denotes an optionally substituted (hetero)aryl group, and X is defined as in general formula (A). 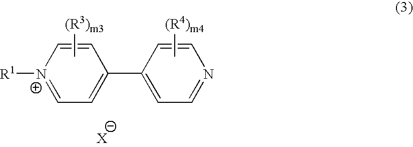
In general formula (3), R¹, R³, R⁴, m3, m4 and X are respectively defined as in general formula (A) or (2).
Ar¹—NH₂ (4)
In general formula (4), Ar¹ is defined as in general formula (A). 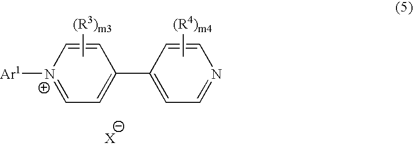
In general formula (5), Ar¹, R³, R⁴, m3, m4 and X are respectively defined as in general formula (A).
R²—X (6)
In general formula (6), R² denotes an optionally substituted (hetero)aryl group, and X is defined as in general formula (A). 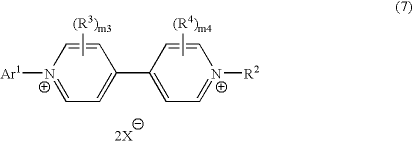
In general formula (7), Ar¹, R², R³, R⁴, m3, m4 and X are respectively defined as in general formula (A) or (6).
Ar²—NH₂ (8)
In general formula (8), Ar² is defined as in general formula (A).

2. The method of manufacturing according to claim 1, wherein Ar¹ and Ar² are different from each other.

3. The method of manufacturing according to claim 1, which comprises no purification step between step (a) and step (b) and between step (c) and step (d).

4. The method of manufacturing according to claim 1, which comprises an extraction step with water between step (b) and step (c).

5. The method of manufacturing according to claim 1, wherein the solvent comprises at least one selected from the group comprising of acetonitrile, an amide solvent, and an alcohol solvent.

6. A method of manufacturing a bipyridinium compound denoted by general formula (7) comprising steps of:
(a) manufacturing a bipyridinium compound denoted by general formula (3) by reacting a bipyridine compound denoted by general formula (1) with a (hetero)arylhalogen compound denoted by general formula (2) in a solvent;
(b) manufacturing a N-aryl-substituted bipyridinium compound denoted by general formula (5) by reacting the bipyridinium compound denoted by general formula (3) that has been obtained in step (a) with an amine compound denoted by general formula (4) in a solvent; and
(c) manufacturing a bipyridinium compound denoted by general formula (7) by reacting the N-aryl-substituted bipyridinium compound denoted by general formula (5) that has been obtained in step (b) with a (hetero)arylhalogen compound denoted by general formula (6) in a solvent. 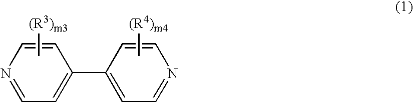
In general formula (1), R³, R⁴, m3 and m4 are respectively defined as in general formula (A).
R¹—X (2)
In general formula (2), R¹ denotes an optionally substituted (hetero)aryl group, and X is defined as in general formula (A). 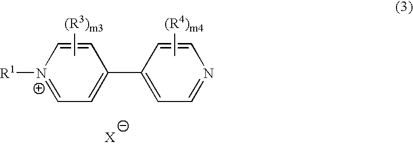
In general formula (3), R¹, R³, R⁴, m3, m4 and X are respectively defined as in general formula (A) or (2).
Ar¹—NH₂ (4)
In general formula (4), Ar¹ is defined as in general formula (A). 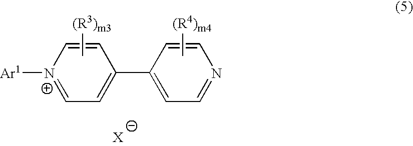
In general formula (5), Ar¹, R³, R⁴, m3, m4 and X are respectively defined as in general formula (A).
R²—X (6)
In general formula (6), R² denotes an optionally substituted (hetero)aryl group, and X is defined as in general formula (A). 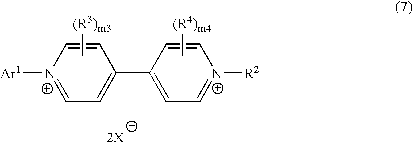
In general formula (7), Ar¹, R², R³, R⁴, m3, m4 and X are respectively defined as in general formula (A) or (6).

7. The method of manufacturing according to claim 6, which comprises an extraction step with water between step (b) and step (c).

8. The method of manufacturing according to claim 6, wherein the solvent comprises at least one selected from the group comprising of acetonitrile, an amide solvent, and an alcohol solvent.

9. A method of manufacturing a bipyridinium compound denoted by general formula (B) comprising steps of:
(a) manufacturing a bipyridinium compound denoted by general formula (3) by reacting a bipyridine compound denoted by general formula (1) with a (hetero)arylhalogen compound denoted by general formula (2) in a solvent;
(b) manufacturing a N-aryl-substituted bipyridinium compound denoted by general formula (5) by reacting the bipyridinium compound denoted by general formula (3) that has been obtained in step (a) with an amine compound denoted by general formula (4) in a solvent;
(c) manufacturing a bipyridinium compound denoted by general formula (7) by reacting the N-aryl-substituted bipyridinium compound denoted by general formula (5) that has been obtained in step (b) with a (hetero)arylhalogen compound denoted by general formula (6) in a solvent; and
(e) manufacturing the bipyridinium compound denoted by general formula (B) by reacting the bipyridinium compound denoted by general formula (7) that has been obtained in step (c) with a diamine compound denoted by general formula (9) in a solvent. 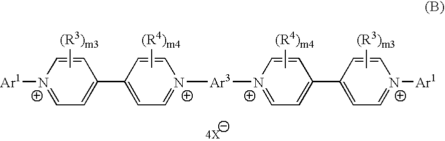
In general formula (B), Ar³ denotes an optionally substituted (hetero)arylene group, and Ar¹, R³, R⁴, m3, m4 and X are respectively defined as in general formula (A). 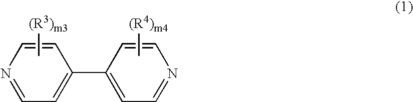
In general formula (1), R³, R⁴, m3 and m4 are respectively defined as in general formula (A).
R¹—X (2)
In general formula (2), R¹ denotes an optionally substituted (hetero)aryl group, and X is defined as in general formula (A). 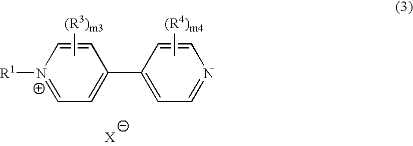
In general formula (3), R¹, R³, R⁴, m3, m4 and X are respectively defined as in general formula (A) or (2).
Ar¹—NH₂ (4)
In general formula (4), Ar¹ is defined as in general formula (A). 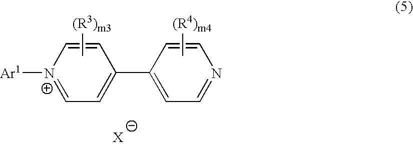
In general formula (5), Ar¹, R³, R⁴, m3, m4 and X are respectively defined as in general formula (A).
R²—X (6)
In general formula (6), R² denotes an optionally substituted (hetero)aryl group, and X is defined as in general formula (A). 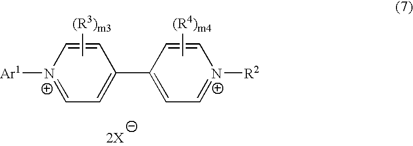
In general formula (7), Ar¹, R², R³, R⁴, m3, m4 and X are respectively defined as in general formula (A) or (6).
H₂N—Ar³—NH₂ (9)
In general formula (9), Ar³ is defined as in general formula (B).

10. The method of manufacturing according to claim 9, which comprises no purification step between step (a) and step (b) and between step (c) and step (e).

11. The method of manufacturing according to claim 9, which comprises an extraction step with water between step (b) and step (c).

12. The method of manufacturing according to claim 9, wherein the solvent comprises at least one selected from the group comprising of acetonitrile, an amide solvent, and an alcohol solvent.

13. A method of manufacturing a dye compound denoted by general formula (D) comprising:
manufacturing a bipyridinium compound denoted by general formula (A) by the manufacturing method according to claim 1; and
manufacturing the dye compound denoted by general formula (D) by reacting the bipyridinium compound that has been obtained with an anionic dye. 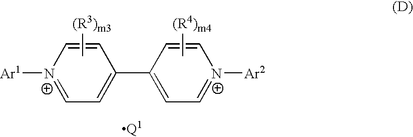
In general formula (D), Q¹ denotes a divalent anionic dye moiety, and Ar¹, Ar², R³, R⁴, m3 and m4 are respectively defined as in general formula (A).

14. The method of manufacturing according to claim 13, wherein the divalent anionic dye moiety is an oxonol dye denoted by general formula (10). 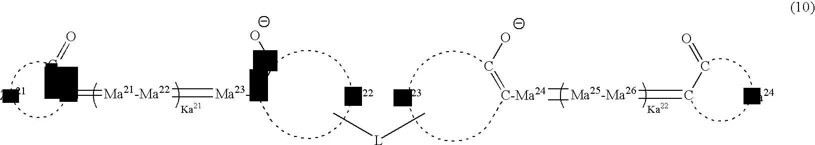
In general formula (10), Za²¹, Za²², Za²³ and Za²⁴ each independently denote an atom group forming an acidic nucleus, Ma²¹, Ma²², Ma²³, Ma²⁴, Ma²⁵ and Ma²⁶ each independently denote a substituted or unsubstituted methine group, L denotes a divalent linking group that does not form a 7r-conjugated system with two bonds, Ka²¹ and Ka²² each independently denote an integer ranging from 0 to 3. When Ka²¹ denotes 2 or 3, plural Ma²¹ and Ma²² present may be identical or different from each other, and when Ka²² denotes 2 or 3, Ma²⁵ and Ma²⁶ present may be identical or different from each other

15. A method of manufacturing a dye compound denoted by general formula (E) comprising:
manufacturing a bipyridiniurn compound denoted by general formula (B) by the manufacturing method according to claim 9; and
manufacturing the dye compound denoted by general formula (E) by reacting the bipyridinium compound that has been obtained with an anionic dye. 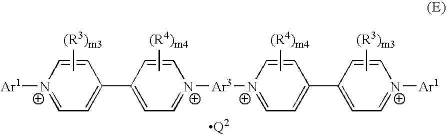
In general formula (E), Q² denotes two divalent anionic dye moieties, and Ar¹, Ar³, R³, R⁴, m3 and m4 are respectively defined as in general formula (B).

16. The method of manufacturing according to claim 15, wherein the divalent anionic dye moiety is an oxonol dye denoted by general formula (10). 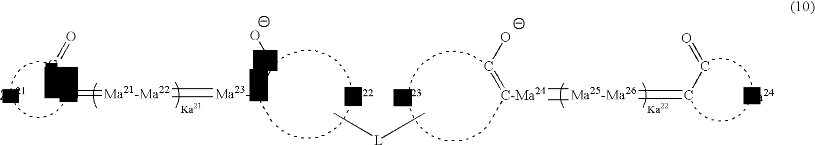
In general formula (10), Za²¹, Za²², Za²³ and Za²⁴ each independently denote an atom group forming an acidic nucleus, Ma²¹, Ma²², Ma²³, Ma²⁴, Ma²⁵ and Ma²⁶ each independently denote a substituted or unsubstituted methine group, L denotes a divalent linking group that does not form a π-conjugated system with two bonds, Ka²¹ and Ka²² each independently denote an integer ranging from 0 to 3. When Ka²¹ denotes 2 or 3, plural Ma²¹ and Ma²² present may be identical or different from each other, and when Ka²² denotes 2 or 3, Ma²⁵ and Ma²⁶ present may be identical or different from each other.

17. A compound denoted by general formula (C) or a salt thereof. 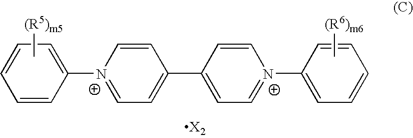
In general formula (C), R⁵ and R⁶ each independently denote a substituent that may form a ring with a benzene ring to which the substituent substitutes, m5 and m6 each independently denote an integer ranging from 0 to 5, and X² denotes an anion that neutralizes a charge within a molecule. When m5 denotes an integer ranging from 2 to 5, plural R⁵ present may be identical or different from each other and when m6 denotes an integer ranging from 2 to 5, plural R⁶ present may be identical or different from each other. m5 and m6 do not both denote 0, and the substituent group denoted by (R⁵)m5 and the substituent group denoted by (R⁶)m6 are not identical.

18. A dye compound denoted by general formula (F). 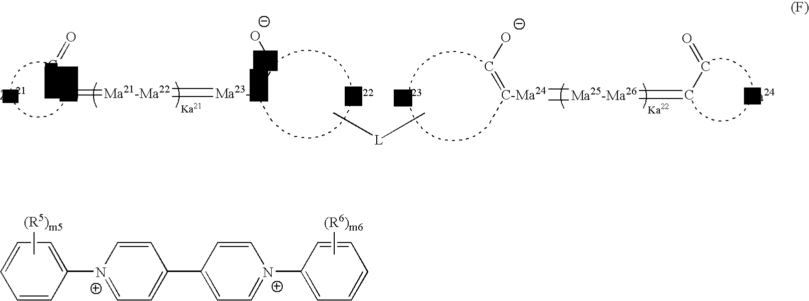
In general formula (F), Za²¹, Za²², Za²³, Za²⁴, Ma²¹, Ma²², M²³, M²⁴, M²⁵, Ma²⁶, L, Ka²¹ and Ka²² are respectively defined as in general formula (10), and R⁵, R⁶, m5 and m6 are respectively defined as in general formula (C).
